SEQUENCE LISTING (55 sequences)

SEQ ID NO: 1 - protein, 6 aa, Mus musculus
GNIHSY

SEQ ID NO: 2 - protein, 4 aa, Mus musculus
NAKT

SEQ ID NO: 3 - protein, 9 aa, Mus musculus
QHFWNTPPT

SEQ ID NO: 4 - protein, 8 aa, Mus musculus
GFSLTSYG

SEQ ID NO: 5 - protein, 7 aa, Mus musculus
IWRGGGT

SEQ ID NO: 6 - protein, 13 aa, Mus musculus
ARSRYDEEESMNY

SEQ ID NO: 7 - protein, 107 aa, synthetic construct
EIVLTQSPSSLSASVGDSVTITCRASGNIHSYLAWYQQKPGKAPKLLIYNAKTLADGVPSRFSGSGSGTQYTLTISSLQPEDFATYYCQHFWNTPPTFGAGTKLELK

SEQ ID NO: 8 - protein, 107 aa, synthetic construct
QIQLTQSPSFLSASVGDSVTITCRASGNIHSYLAWYQQKPGKAPQLLIYNAKTLADGVPSRFSGSGSGTEYTLTISSLQPEDFATYYCQHFWNTPPTFGAGTKLELK

SEQ ID NO: 9 - protein, 107 aa, synthetic construct
EIVLTQSPATLSLSPGERATLSCRASGNIHSYLAWYQQKPGQAPRLLIYNAKTLADGIPARFSGSGSGTDYTLTISSLEPEDFASYYCQHFWNTPPTFGAGTKLELK

SEQ ID NO: 10 - protein, 107 aa, synthetic construct
EIVLTQSPGTLSLSPGERATLSCRASGNIHSYLAWYQQKPGQAPRLLIYNAKTLADGIPDRFSGSGSGTDYTLTISRLEPEDFAVYYCQHFWNTPPTFGAGTKLELK

SEQ ID NO: 11 - protein, 119 aa, synthetic construct
QVQLQESGPGLVKPSETLSLTCTVSGFSLTSYGIHWIRQSPGRGLEWIGVIWRGGGTDSNAAFMSRVTISVDTSKSQVSLKLGSVTAADTAIYYCARSRYDEEESMNYWGQGTSVTVSS

SEQ ID NO: 12 - protein, 119 aa, synthetic construct
QVQLQESGPGLVKPSETLSLTCTVSGFSLTSYGIHWIRQSPGRGLEWIGVIWRGGGTDSNAAFMSRITISRDTSKTQVSLKLGSVTAADTAIYYCARSRYDEEESMNYWGQGTSVTVSS

SEQ ID NO: 13 - protein, 246 aa, synthetic construct
EIVLTQSPSSLSASVGDSVTITCRASGNIHSYLAWYQQKPGKAPKLLIYNAKTLADGVPSRFSGSGSGTQYTLTISSLQPEDFATYYCQHFWNTPPTFGAGTKLELKGGGGSGGGGSGGGGSGGGGSQVQLQESGPGLVKPSETLSLTCTVSGFSLTSYGIHWIRQSPGRGLEWIGVIWRGGGTDSNAAFMSRVTISVDTSKSQVSLKLGSVTAADTAIYYCARSRYDEEESMNYWGQGTSVTVSS

SEQ ID NO: 14 - protein, 246 aa, synthetic construct
EIVLTQSPSSLSASVGDSVTITCRASGNIHSYLAWYQQKPGKAPKLLIYNAKTLADGVPSRFSGSGSGTQYTLTISSLQPEDFATYYCQHFWNTPPTFGAGTKLELKGGGGSGGGGSGGGGSGGGGSQVQLQESGPGLVKPSETLSLTCTVSGFSLTSYGIHWIRQSPGRGLEWIGVIWRGGGTDSNAAFMSRITISRDTSKTQVSLKLGSVTAADTAIYYCARSRYDEEESMNYWGQGTSVTVSS

SEQ ID NO: 15 - protein, 246 aa, synthetic construct
QIQLTQSPSFLSASVGDSVTITCRASGNIHSYLAWYQQKPGKAPQLLIYNAKTLADGVPSRFSGSGSGTEYTLTISSLQPEDFATYYCQHFWNTPPTFGAGTKLELKGGGGSGGGGSGGGGSGGGGSQVQLQESGPGLVKPSETLSLTCTVSGFSLTSYGIHWIRQSPGRGLEWIGVIWRGGGTDSNAAFMSRVTISVDTSKSQVSLKLGSVTAADTAIYYCARSRYDEEESMNYWGQGTSVTVSS

SEQ ID NO: 16 - protein, 246 aa, synthetic construct
QIQLTQSPSFLSASVGDSVTITCRASGNIHSYLAWYQQKPGKAPQLLIYNAKTLADGVPSRFSGSGSGTEYTLTISSLQPEDFATYYCQHFWNTPPTFGAGTKLELKGGGGSGGGGSGGGGSGGGGSQVQLQESGPGLVKPSETLSLTCTVSGFSLTSYGIHWIRQSPGRGLEWIGVIWRGGGTDSNAAFMSRITISRDTSKTQVSLKLGSVTAADTAIYYCARSRYDEEESMNYWGQGTSVTVSS

SEQ ID NO: 17 - protein, 246 aa, synthetic construct
EIVLTQSPATLSLSPGERATLSCRASGNIHSYLAWYQQKPGQAPRLLIYNAKTLADGIPARFSGSGSGTDYTLTISSLEPEDFASYYCQHFWNTPPTFGAGTKLELKGGGGSGGGGSGGGGSGGGGSQVQLQESGPGLVKPSETLSLTCTVSGFSLTSYGIHWIRQSPGRGLEWIGVIWRGGGTDSNAAFMSRVTISVDTSKSQVSLKLGSVTAADTAIYYCARSRYDEEESMNYWGQGTSVTVSS

SEQ ID NO: 18 - protein, 246 aa, synthetic construct
EIVLTQSPATLSLSPGERATLSCRASGNIHSYLAWYQQKPGQAPRLLIYNAKTLADGIPARFSGSGSGTDYTLTISSLEPEDFASYYCQHFWNTPPTFGAGTKLELKGGGGSGGGGSGGGGSGGGGSQVQLQESGPGLVKPSETLSLTCTVSGFSLTSYGIHWIRQSPGRGLEWIGVIWRGGGTDSNAAFMSRITISRDTSKTQVSLKLGSVTAADTAIYYCARSRYDEEESMNYWGQGTSVTVSS

SEQ ID NO: 19 - protein, 246 aa, synthetic construct
EIVLTQSPGTLSLSPGERATLSCRASGNIHSYLAWYQQKPGQAPRLLIYNAKTLADGIPDRFSGSGSGTDYTLTISRLEPEDFAVYYCQHFWNTPPTFGAGTKLELKGGGGSGGGGSGGGGSGGGGSQVQLQESGPGLVKPSETLSLTCTVSGFSLTSYGIHWIRQSPGRGLEWIGVIWRGGGTDSNAAFMSRVTISVDTSKSQVSLKLGSVTAADTAIYYCARSRYDEEESMNYWGQGTSVTVSS

SEQ ID NO: 20 - protein, 246 aa, synthetic construct
EIVLTQSPGTLSLSPGERATLSCRASGNIHSYLAWYQQKPGQAPRLLIYNAKTLADGIPDRFSGSGSGTDYTLTISRLEPEDFAVYYCQHFWNTPPTFGAGTKLELKGGGGSGGGGSGGGGSGGGGSQVQLQESGPGLVKPSETLSLTCTVSGFSLTSYGIHWIRQSPGRGLEWIGVIWRGGGTDSNAAFMSRITISRDTSKTQVSLKLGSVTAADTAIYYCARSRYDEEESMNYWGQGTSVTVSS

SEQ ID NO: 21 - DNA, 18 bp, Mus musculus
  source 1..18: mol_type = genomic DNA
ggcaacatccactcctac

SEQ ID NO: 22 - DNA, 12 bp, Mus musculus
  source 1..12: mol_type = genomic DNA
aacgccaagacc

SEQ ID NO: 23 - DNA, 27 bp, Mus musculus
  source 1..27: mol_type = genomic DNA
accccccccaccttcggcgccggcacc

SEQ ID NO: 24 - DNA, 24 bp, Mus musculus
  source 1..24: mol_type = genomic DNA
ggcatccactggatccggcagtcc

SEQ ID NO: 25 - DNA, 21 bp, Mus musculus
  source 1..21: mol_type = genomic DNA
gactccaacgccgccttcatg

SEQ ID NO: 26 - DNA, 39 bp, Mus musculus
  source 1..39: mol_type = genomic DNA
gcccggtcccggtacgacgaggaggagtccatgaactac

SEQ ID NO: 27 - DNA, 321 bp, synthetic construct
  source 1..321: mol_type = other DNA
gagatcgtgctgacccagtccccctcctccctgtccgcctccgtgggcgactccgtgaccatcacctgccgggcctccggcaacatccactcctacctggcctggtaccagcagaagcccggcaaggcccccaagctgctgatctacaacgccaagaccctggccgacggcgtgccctcccggttctccggctccggctccggcacccagtacaccctgaccatctcctccctgcagcccgaggacttcgccacctactactgccagcacttctggaacaccccccccaccttcggcgccggcaccaagctggagctgaag

SEQ ID NO: 28 - DNA, 321 bp, synthetic construct
  source 1..321: mol_type = other DNA
cagatccagctgacccagtccccctccttcctgtccgcctccgtgggcgactccgtgaccatcacctgccgggcctccggcaacatccactcctacctggcctggtaccagcagaagcccggcaaggccccccagctgctgatctacaacgccaagaccctggccgacggcgtgccctcccggttctccggctccggctccggcaccgagtacaccctgaccatctcctccctgcagcccgaggacttcgccacctactactgccagcacttctggaacaccccccccaccttcggcgccggcaccaagctggagctgaag

SEQ ID NO: 29 - DNA, 321 bp, synthetic construct
  source 1..321: mol_type = other DNA
gagatcgtgctgacccagtcccccgccaccctgtccctgtcccccggcgagcgggccaccctgtcctgccgggcctccggcaacatccactcctacctggcctggtaccagcagaagcccggccaggccccccggctgctgatctacaacgccaagaccctggccgacggcatccccgcccggttctccggctccggctccggcaccgactacaccctgaccatctcctccctggagcccgaggacttcgcctcctactactgccagcacttctggaacaccccccccaccttcggcgccggcaccaagctggagctgaag

SEQ ID NO: 30 - DNA, 321 bp, synthetic construct
  source 1..321: mol_type = other DNA
gagatcgtgctgacccagtcccccggcaccctgtccctgtcccccggcgagcgggccaccctgtcctgccgggcctccggcaacatccactcctacctggcctggtaccagcagaagcccggccaggccccccggctgctgatctacaacgccaagaccctggccgacggcatccccgaccggttctccggctccggctccggcaccgactacaccctgaccatctcccggctggagcccgaggacttcgccgtgtactactgccagcacttctggaacaccccccccaccttcggcgccggcaccaagctggagctgaag

SEQ ID NO: 31 - DNA, 357 bp, synthetic construct
  source 1..357: mol_type = other DNA
caggtgcagctgcaggagtccggccccggcctggtgaagccctccgagaccctgtccctgacctgcaccgtgtccggcttctccctgacctcctacggcatccactggatccggcagtcccccggccggggcctggagtggatcggcgtgatctggcggggcggcggcaccgactccaacgccgccttcatgtcccgggtgaccatctccgtggacacctccaagtcccaggtgtccctgaagctgggctccgtgaccgccgccgacaccgccatctactactgcgcccggtcccggtacgacgaggaggagtccatgaactactggggccagggcacctccgtgaccgtgtcctcc

SEQ ID NO: 32 - DNA, 357 bp, synthetic construct
  source 1..357: mol_type = other DNA
caggtgcagctgcaggagtccggccccggcctggtgaagccctccgagaccctgtccctgacctgcaccgtgtccggcttctccctgacctcctacggcatccactggatccggcagtcccccggccggggcctggagtggatcggcgtgatctggcggggcggcggcaccgactccaacgccgccttcatgtcccggatcaccatctcccgggacacctccaagacccaggtgtccctgaagctgggctccgtgaccgccgccgacaccgccatctactactgcgcccggtcccggtacgacgaggaggagtccatgaactactggggccagggcacctccgtgaccgtgtcctcc

SEQ ID NO: 33 - DNA, 738 bp, synthetic construct
  source 1..738: mol_type = other DNA
gagatcgtgctgacccagtccccctcctccctgtccgcctccgtgggcgactccgtgaccatcacctgccgggcctccggcaacatccactcctacctggcctggtaccagcagaagcccggcaaggcccccaagctgctgatctacaacgccaagaccctggccgacggcgtgccctcccggttctccggctccggctccggcacccagtacaccctgaccatctcctccctgcagcccgaggacttcgccacctactactgccagcacttctggaacaccccccccaccttcggcgccggcaccaagctggagctgaagggcggcggcggctccggcggcggcggctccggcggcggcggctccggcggcggcggctcccaggtgcagctgcaggagtccggccccggcctggtgaagccctccgagaccctgtccctgacctgcaccgtgtccggcttctccctgacctcctacggcatccactggatccggcagtcccccggccggggcctggagtggatcggcgtgatctggcggggcggcggcaccgactccaacgccgccttcatgtcccgggtgaccatctccgtggacacctccaagtcccaggtgtccctgaagctgggctccgtgaccgccgccgacaccgccatctactactgcgcccggtcccggtacgacgaggaggagtccatgaactactggggccagggcacctccgtgaccgtgtcctcc

SEQ ID NO: 34 - DNA, 738 bp, synthetic construct
  source 1..738: mol_type = other DNA
gagatcgtgctgacccagtccccctcctccctgtccgcctccgtgggcgactccgtgaccatcacctgccgggcctccggcaacatccactcctacctggcctggtaccagcagaagcccggcaaggcccccaagctgctgatctacaacgccaagaccctggccgacggcgtgccctcccggttctccggctccggctccggcacccagtacaccctgaccatctcctccctgcagcccgaggacttcgccacctactactgccagcacttctggaacaccccccccaccttcggcgccggcaccaagctggagctgaagggcggcggcggctccggcggcggcggctccggcggcggcggctccggcggcggcggctcccaggtgcagctgcaggagtccggccccggcctggtgaagccctccgagaccctgtccctgacctgcaccgtgtccggcttctccctgacctcctacggcatccactggatccggcagtcccccggccggggcctggagtggatcggcgtgatctggcggggcggcggcaccgactccaacgccgccttcatgtcccggatcaccatctcccgggacacctccaagacccaggtgtccctgaagctgggctccgtgaccgccgccgacaccgccatctactactgcgcccggtcccggtacgacgaggaggagtccatgaactactggggccagggcacctccgtgaccgtgtcctcc

SEQ ID NO: 35 - DNA, 738 bp, synthetic construct
  source 1..738: mol_type = other DNA
cagatccagctgacccagtccccctccttcctgtccgcctccgtgggcgactccgtgaccatcacctgccgggcctccggcaacatccactcctacctggcctggtaccagcagaagcccggcaaggccccccagctgctgatctacaacgccaagaccctggccgacggcgtgccctcccggttctccggctccggctccggcaccgagtacaccctgaccatctcctccctgcagcccgaggacttcgccacctactactgccagcacttctggaacaccccccccaccttcggcgccggcaccaagctggagctgaagggcggcggcggctccggcggcggcggctccggcggcggcggctccggcggcggcggctcccaggtgcagctgcaggagtccggccccggcctggtgaagccctccgagaccctgtccctgacctgcaccgtgtccggcttctccctgacctcctacggcatccactggatccggcagtcccccggccggggcctggagtggatcggcgtgatctggcggggcggcggcaccgactccaacgccgccttcatgtcccgggtgaccatctccgtggacacctccaagtcccaggtgtccctgaagctgggctccgtgaccgccgccgacaccgccatctactactgcgcccggtcccggtacgacgaggaggagtccatgaactactggggccagggcacctccgtgaccgtgtcctcc

SEQ ID NO: 36 - DNA, 738 bp, synthetic construct
  source 1..738: mol_type = other DNA
cagatccagctgacccagtccccctccttcctgtccgcctccgtgggcgactccgtgaccatcacctgccgggcctccggcaacatccactcctacctggcctggtaccagcagaagcccggcaaggccccccagctgctgatctacaacgccaagaccctggccgacggcgtgccctcccggttctccggctccggctccggcaccgagtacaccctgaccatctcctccctgcagcccgaggacttcgccacctactactgccagcacttctggaacaccccccccaccttcggcgccggcaccaagctggagctgaagggcggcggcggctccggcggcggcggctccggcggcggcggctccggcggcggcggctcccaggtgcagctgcaggagtccggccccggcctggtgaagccctccgagaccctgtccctgacctgcaccgtgtccggcttctccctgacctcctacggcatccactggatccggcagtcccccggccggggcctggagtggatcggcgtgatctggcggggcggcggcaccgactccaacgccgccttcatgtcccggatcaccatctcccgggacacctccaagacccaggtgtccctgaagctgggctccgtgaccgccgccgacaccgccatctactactgcgcccggtcccggtacgacgaggaggagtccatgaactactggggccagggcacctccgtgaccgtgtcctcc

SEQ ID NO: 37 - DNA, 738 bp, synthetic construct
  source 1..738: mol_type = other DNA
gagatcgtgctgacccagtcccccgccaccctgtccctgtcccccggcgagcgggccaccctgtcctgccgggcctccggcaacatccactcctacctggcctggtaccagcagaagcccggccaggccccccggctgctgatctacaacgccaagaccctggccgacggcatccccgcccggttctccggctccggctccggcaccgactacaccctgaccatctcctccctggagcccgaggacttcgcctcctactactgccagcacttctggaacaccccccccaccttcggcgccggcaccaagctggagctgaagggcggcggcggctccggcggcggcggctccggcggcggcggctccggcggcggcggctcccaggtgcagctgcaggagtccggccccggcctggtgaagccctccgagaccctgtccctgacctgcaccgtgtccggcttctccctgacctcctacggcatccactggatccggcagtcccccggccggggcctggagtggatcggcgtgatctggcggggcggcggcaccgactccaacgccgccttcatgtcccgggtgaccatctccgtggacacctccaagtcccaggtgtccctgaagctgggctccgtgaccgccgccgacaccgccatctactactgcgcccggtcccggtacgacgaggaggagtccatgaactactggggccagggcacctccgtgaccgtgtcctcc

SEQ ID NO: 38 - DNA, 738 bp, synthetic construct
  source 1..738: mol_type = other DNA
gagatcgtgctgacccagtcccccgccaccctgtccctgtcccccggcgagcgggccaccctgtcctgccgggcctccggcaacatccactcctacctggcctggtaccagcagaagcccggccaggccccccggctgctgatctacaacgccaagaccctggccgacggcatccccgcccggttctccggctccggctccggcaccgactacaccctgaccatctcctccctggagcccgaggacttcgcctcctactactgccagcacttctggaacaccccccccaccttcggcgccggcaccaagctggagctgaagggcggcggcggctccggcggcggcggctccggcggcggcggctccggcggcggcggctcccaggtgcagctgcaggagtccggccccggcctggtgaagccctccgagaccctgtccctgacctgcaccgtgtccggcttctccctgacctcctacggcatccactggatccggcagtcccccggccggggcctggagtggatcggcgtgatctggcggggcggcggcaccgactccaacgccgccttcatgtcccggatcaccatctcccgggacacctccaagacccaggtgtccctgaagctgggctccgtgaccgccgccgacaccgccatctactactgcgcccggtcccggtacgacgaggaggagtccatgaactactggggccagggcacctccgtgaccgtgtcctcc

SEQ ID NO: 39 - DNA, 738 bp, synthetic construct
  source 1..738: mol_type = other DNA
gagatcgtgctgacccagtcccccggcaccctgtccctgtcccccggcgagcgggccaccctgtcctgccgggcctccggcaacatccactcctacctggcctggtaccagcagaagcccggccaggccccccggctgctgatctacaacgccaagaccctggccgacggcatccccgaccggttctccggctccggctccggcaccgactacaccctgaccatctcccggctggagcccgaggacttcgccgtgtactactgccagcacttctggaacaccccccccaccttcggcgccggcaccaagctggagctgaagggcggcggcggctccggcggcggcggctccggcggcggcggctccggcggcggcggctcccaggtgcagctgcaggagtccggccccggcctggtgaagccctccgagaccctgtccctgacctgcaccgtgtccggcttctccctgacctcctacggcatccactggatccggcagtcccccggccggggcctggagtggatcggcgtgatctggcggggcggcggcaccgactccaacgccgccttcatgtcccgggtgaccatctccgtggacacctccaagtcccaggtgtccctgaagctgggctccgtgaccgccgccgacaccgccatctactactgcgcccggtcccggtacgacgaggaggagtccatgaactactggggccagggcacctccgtgaccgtgtcctcc

SEQ ID NO: 40 - DNA, 738 bp, synthetic construct
  source 1..738: mol_type = other DNA
gagatcgtgctgacccagtcccccggcaccctgtccctgtcccccggcgagcgggccaccctgtcctgccgggcctccggcaacatccactcctacctggcctggtaccagcagaagcccggccaggccccccggctgctgatctacaacgccaagaccctggccgacggcatccccgaccggttctccggctccggctccggcaccgactacaccctgaccatctcccggctggagcccgaggacttcgccgtgtactactgccagcacttctggaacaccccccccaccttcggcgccggcaccaagctggagctgaagggcggcggcggctccggcggcggcggctccggcggcggcggctccggcggcggcggctcccaggtgcagctgcaggagtccggccccggcctggtgaagccctccgagaccctgtccctgacctgcaccgtgtccggcttctccctgacctcctacggcatccactggatccggcagtcccccggccggggcctggagtggatcggcgtgatctggcggggcggcggcaccgactccaacgccgccttcatgtcccggatcaccatctcccgggacacctccaagacccaggtgtccctgaagctgggctccgtgaccgccgccgacaccgccatctactactgcgcccggtcccggtacgacgaggaggagtccatgaactactggggccagggcacctccgtgaccgtgtcctcc

SEQ ID NO: 41 - protein, 110 aa, Homo sapiens
SYELTQPPSVSVAPGKTARITCAGNNIGSKSVHWYQQKPGQAPVLVIYYDSDRPSGIPERFSGSNSGNTATLTISRVEAGDEADYYCQVWDSGSDHPWVFGGGTKLTVLR

SEQ ID NO: 42 - protein, 116 aa, Homo sapiens
EVQLVESGGGLVKPGGSLRLSCAASGFTFRSYSMNWVRQAPGKGLEWVSSIISSSSYIYYADSVKGRFTISRDNAKNSLYLQMNSLRAEDTALYYCARDQNAMDVWGQGTTVTVSS

SEQ ID NO: 43 - protein, 215 aa, Homo sapiens
SYELTQPPSVSVAPGKTARITCAGNNIGSKSVHWYQQKPGQAPVLVIYYDSDRPSGIPERFSGSNSGNTATLTISRVEAGDEADYYCQVWDSGSDHPWVFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 44 - protein, 107 aa, Mus musculus
QIVLTQSPASLSASVGETVTITCRASGNIHSYLAWYQQKQGKSPQLLVYNAKTLADGVPSRFSGSGSGTQYSLKINSLQPEDFGSYYCQHFWNTPPTFGAGTKLELK

SEQ ID NO: 45 - protein, 119 aa, Mus musculus
QVQLQQSGPGLVQPSQSLSITCTVSGFSLTSYGIHWVRQSPGKGLEWLGVIWRGGGTDSNAAFMSRLSITKDNSKSQVFFKMNSLQADDTAIYYCARSRYDEEESMNYWGQGTSVTVSS

SEQ ID NO: 46 - protein, 20 aa, synthetic construct
GGGGSGGGGSGGGGSGGGGS

SEQ ID NO: 47 - DNA, 705 bp, synthetic construct
  source 1..705: mol_type = other DNA
atggcctggaccgttctcctcctcggcctcctctctcactgcacaggctctgtgacctcctatgtgctgactcagccaccctcagtgtcagtggccccaggaaagacggccaggattacctgtgggggaaacaacattggaagtaaaagtgtgcactggtaccagcagaagccaggccaggcccctgtgctggtcatctattatgatagcgaccggccctcagggatccctgagcgattctctggctccaagtctgggaacacggccaccctgaccatcagcagggtcgaagccggggatgaggccgactattactgtcaggtgtgggatagtagtagtgatcatccttgggtgttcggcggagggaccaagctgaccgtcctacggcagcccaaggccaaccccacggtcactctgttcccgccctcctctgaggagctccaagccaacaaggccacactagtgtgtctgatcagtgacttctacccgggagctgtgacagtggcttggaaggcagatggcagccccgtcaaggcgggagtggagacgaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcatag

SEQ ID NO: 48 - DNA, 8581 bp, synthetic construct
  source 1..8581: mol_type = other DNA
gttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgccgccaccgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagcggccgctctagacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctg

SEQ ID NO: 49 - DNA, 9232 bp, synthetic construct
  source 1..9232: mol_type = other DNA
gttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggatctacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggggatcaattgcatgaagaatctgcttagggttaggcgttttgcgctgcttcgcgaggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacggctagcgccgccaccgaatctaagtacggaccgccctgccccccttgccctgcccccgagttcctgggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttcaacagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagtccctgtctctgagcctgggcaagatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggccctaggatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgtgagcggccgctctagacccgggctgcaggaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagccgtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagaattcgatatcaagcttatcgataccgtcgacctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccaccgcggtggcggcctcgaggtcgagatccggtcgaccagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttcgacggtatcgattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggaattcggagtggcgagccctcagatcctgcatataagcagctgctttttgcctgtactgggtctctctg

SEQ ID NO: 50 - DNA, 990 bp, Homo sapiens
  source 1..990: mol_type = genomic DNA
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaag

SEQ ID NO: 51 - DNA, 321 bp, Homo sapiens
  source 1..321: mol_type = genomic DNA
cgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgc

SEQ ID NO: 52 - protein, 569 aa, Homo sapiens
EVQLVESGGGLVKPGGSLRLSCAASGFTFSGYSMNWVRQAPGKGLEWVSSISSSSTYIYYVDSVRGRFTISRDNAKNSLYLQMNSLRAEDTAVYYCARDENDMDVWGQGTTVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 53 - protein, 215 aa, Homo sapiens
SYVLTQPPSVSVAPGKTARITCGGNNIGTKSVHWYQQKPGQAPVLVIYYDSDRPSGIPERFSGSNSGNTATLTISRVEAGDEADYYCQVWDSGSDHPWVFGGGTKLTVLRQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 54 - protein, 95 aa, Homo sapiens
DIQLTQSPSFLSASVGDRVTITCRASQGISSYLAWYQQKPGKAPKLLIYAASTLQSGVPSRFSGSGSGTEFTLTISSLQPEDFATYYCQQLNSYP

SEQ ID NO: 55 - protein, 97 aa, Homo sapiens
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAR

(19) United States
(12) Patent Application Publication
(10) Pub. No.: US 2026/0035459 A1
(43) Pub. Date: Feb. 5, 2026

(54) HUMANIZED CHIMERIC ANTIGEN RECEPTORS TARGETING THE B-CELL RECEPTOR OF CHRONIC LYMPHOCYTIC LEUKEMIA AND USES THEREOF

(71) Applicant: AVA LIFESCIENCE GMBH, Bad Krozingen (DE)

(72) Inventors: Marcus DÜHREN-VON MINDEN, Müllheim (DE); Mascha BINDER, Freiburg (DE)

(73) Assignee: AVA LIFESCIENCE GMBH, Bad Krozingen (DE)

(21) Appl. No.: 18/998,354

(22) PCT Filed: Jul. 25, 2023
(86) PCT No.: PCT/EP2023/070651

§ 371 Date: Jan. 24, 2025

(30) Foreign Application Priority Data
Jul. 25, 2022 (EP) 22186810.2

(51) Int. Cl. C07K 16/28; A61K 40/11; A61K 40/31; A61K 40/42; A61P 35/02; C12N 5/0783

(52) U.S. Cl. CPC C07K 16/2803; A61K 40/11; A61K 40/31; A61K 40/421; A61P 35/02; C12N 5/0636; C07K 2317/24; C07K 2317/622; C07K 2317/73; C07K 2317/92; C07K 2319/03; C12N 2510/00

(57) ABSTRACT

The present invention provides humanized chimeric antigen receptors (CARs) targeting the B-cell receptor (BCR) of CLL cells characterised by R110-mutated immunoglobulin lambda variable 3-21 (IGLV3-21R¹¹⁰).

The Invention also provides nucleic acid sequences encoding the forgoing CARs, vectors containing the same, pharmaceutical compositions and kits with instructions for use.

The present invention provides humanized chimeric antigen receptors (CARs) for the treatment of chronic lymphocytic leukemia (CLL). These CARs target the B-cell receptor (BCR) of CLL cells characterised by R110-mutated immunoglobulin lambda variable 3-21 (IGLV3-21R¹¹⁰).

The invention also provides nucleic acid sequences encoding the forgoing CARs, vectors containing the same, cells expressing the same, pharmaceutical compositions and kits with instructions for use.

### BACKGROUND OF THE INVENTION

Adoptive immunotherapy with chimeric antigen receptor (CAR) redirected immuno effector cells, is emerging as a highly promising approach for the treatment of Leukemias and Lymphomas originating from malignant transformations of B-lineage cells. Since the approval of treatments with CAR-T cells involving the use of a CAR directed against the common B-cell antigen “CD19”, response rates, long-term outcomes, and life-quality of patients with certain types of leukemias and lymphomas of the B-cell type such as acute lymphoblastic leukemia, diffuse large B-cell lymphoma, and mantle cell lymphoma have remarkably improved (for e.g. Maude S L, Frey N, Shaw P A et al. Chimeric Antigen Receptor T Cells for Sustained Remissions in Leukemia; N Engl J Med 2014; 371:1507-15017).

The most common type of leukemia in western countries is chronic lymphocytic leukemia typically occurring in elderly patients, with a two-fold increased risk of developing CLL for men compared to women (Kipps T J, Stevenson F K, Wu C J, Croce C M, Packham G, Wierda W G, et al. Chronic lymphocytic leukemia. Nat Rev Dis Primers (2017) 3:1-12). CLL is a heterogeneous, B-lymphocyte-derived malignancy resulting from the clonal proliferation of a CD5-positive subpopulation of B lymphocytes which progressively accumulate in the bone marrow, lymph nodes, peripheral blood and spleen (Rozman C, Montserrat E. Chronic lymphocytic leukemia. N Engl J Med. 1995; 333:1052-1057).

Clinical and biological evidence has shown that the BCR is one of the major factors in clonal selection and survival of CLL cells as reviewed by Burger and Chiorazzi (Burger J A, Chiorazzi N. B cell receptor signaling in chronic lymphocytic leukemia. Trends Immunol 2013; 34:592-601).

The BCR is a multiprotein structure that is composed of an antigen binding subunit and a signaling subunit, which are non-covalently associated. The antigen-binding subunit consists of a membrane immunoglobulin containing two identical heavy chains and two identical light chains with one constant domain in each light chain and three in each heavy chain. Each heavy chain associates with a light chain to form an antigen-binding site. Each light and each heavy chain contain a variable domain forming the antigen-binding site. The immunoglobulin genes encoded in the Igh, Igl and Igk loci contain large numbers of V (variable), D (diversity) and J (joining) gene segments upstream of one or more constant exons. In a developing B cell, immunoglobulin gene rearrangement randomly assembles V, D and J gene segments to create a complete V exon in the Igh locus, and V and J gene segments in either the Igk or Igl locus. Through combinatorial joining of gene segments, junctional diversity and random heavy and light chain pairing, each individual B cell progenitor generates its own and nearly unique antigen binding subunit, whose antigen-binding affinity can be further refined by somatic hypermutation (SHM).

The BCR's signal transduction moiety is composed of a disulfide-linked heterodimer of the Igα and Igβ (CD79a/CD79b) proteins. Igα and Igβ each contain a single immunoreceptor tyrosine-based activation motif (ITAM) within their cytoplasmic tail that initiates signal transduction following BCR aggregation upon antigen-binding (Flaswinkel, H., Reth, M., 1994. Dual role of the tyrosine activation motif of the Ig-alpha protein during signal transduction via the B cell antigen receptor. EMBO J. 13, 83-89).

Antigen-binding rapidly activates the Src family kinase Lyn leading to the phosphorylation of Iga/lgB. This initiates the formation of a large signaling complex on the cytoplasmic side of the membrane composed of the BCR, various tyrosine kinases, adaptor proteins, and signaling enzymes. Proximal BCR signaling is mediated by the protein tyrosine kinase Syk (spleen tyrosine kinase), which is recruited to the phosphorylated ITAMs of Igα and Igβ, leading to the propagation of the signal via association of Syk with the adaptor protein SLP65 and its downstream signaling enzymes Bruton's tyrosine kinase (BTK) and phospholipase Cγ2 (PLCγ2). Signals emanating the signaling complex activate downstream pathways, including calcium mobilization, phosphoinositide 3-kinases (PI3Ks), nuclear factor-κB (NF-κB), nuclear factor of activated T-cells (NF-AT), mitogen-activated protein kinases (MAPKs), and Rat sarcoma (RAS) signaling pathways (Burger J A and Chiorazzi N, 2013, s.a).

Chronic activation of mature B cells through the B-cell receptor has been shown to be a key process in the formation and development of CLL (Stevenson F K, Krysov S, Davies A J, Steele A J, Packham G. B-cell receptor signaling in chronic lymphocytic leukemia Blood. 2011; 118:4313-4320). This agrees also with a study using EBV protein LMP2A as a constitutively active BCR surrogate, which showed that the development of a mouse B1 subset was dependent on a strong and prolonged BCR stimulation (Casola S, Otipoby K I, Alimzhanov M, et al. B cell receptor signal strength determines B cell fate. Nat Immunol 2004; 5:317-27). Moreover, antigen-independent autonomous signalling of primary CLL B cells due to interactions of two neighbouring BCRs on a cell has been identified as a crucial driver of CLL development, resulting in elevated tyrosine phosphorylation of the BCR proximal signalling molecules, leading to periodic signalling and elevated Ca²⁺ mobilization (Duhren-von Minden M et al. Chronic lymphocytic leukemia is driven by antigen-independent cell-autonomous signalling. Nature. 2012; 489:309-313).

It is well documented that protein kinase Syk is constitutively phosphorylated through sustained BCR signalling, and several studies have revealed that also other key molecules of the signalling pathways downstream of BCR engagement in normal B cells, such as PKC, phosphoinositide 3-kinase and mitogen-activated protein kinase p38, are constitutively activated in B-CLL cells, resulting in the deregulation of the activity or expression of several prosurvival molecules and downstream pathways (Gobessi S, Laurenti L, Longo P G, Carsetti L, Berno V, Sica S et al. Inhibition of constitutive and BCR-induced Syk activation downregulates Mcl-1 and induces apoptosis in chronic lymphocytic leukemia B cells. Leukemia 2009; 23:686-697. Ringshausen I, Schneller F, Bogner C, Hipp S, Duyster J, Peschel C et al. Constitutively activated phosphatidylinositol-3 kinase (PI-3K) is involved in the defect of apoptosis in B-CLL: association with protein kinase C delta. Blood 2002; 100:3741-3748. Plate J M. PI3-kinase regulates survival of chronic lymphocytic leukemia B-cells by preventing caspase 8 activation. Leuk Lymphoma 2004; 45:1519-1529. Sainz-Perez A, Gary-Gouy H, Portier A, Davi F, Merle-Beral H, Galanaud P et al. High Mda-7 expression promotes malignant cell survival and p38 MAP kinase activation in chronic lymphocytic leukemia. Leukemia 2006; 20:498-504.).

Constitutively activated signalling pathways such as NF—KB or PI3K/AKT have been shown to lead to the transcription and overexpression of key antiapoptotic proteins, notably several members of the B-cell lymphoma 2 (Bcl-2) and Inhibitor of Apoptosis Protein (IAP) families (Loeder S et al. A novel paradigm to trigger apoptosis in chronic lymphocytic leukemia. Cancer Res. 2009; 69:8977-8986). It is well established that in addition to Bcl-2 itself, Mcl-1 is a crucial player in impaired apoptosis in CLL cells, and BCR signals reportedly upregulate Mcl-1 expression through the PI3K/AKT pathway (Petlickovski A, Laurenti L, Li X, Marietti S, Chiusolo P, Sica S, Leone G, Efremov D G. Sustained signalling through the B-cell receptor induces Mcl-1 and promotes survival of chronic lymphocytic leukemia B cells. Blood. 2005; 105:4820-4827).

Different aspects of the BCR have been recognized to identify main CLL disease subtypes. For example, the level of somatic hypermutations within the variable region of the BCR immunoglobulin heavy chain (IGHV) has been used as a prognostic marker for decades. CLL patients with a mutated IGHV-gene (M-CLL), i.e. showing less than 98% IGHV gene identity with its closest germline, generally have a more indolent disease course than CLL patients with an unmutated IGHV gene with a germline identity equal to or above 98% (U-CLL). However, exceptions to this rule have been observed in which the mutational IGHV-gene status could not be correlated with a certain disease course. For example, cases using the IGHV3-21-gene, although mostly expressing a mutated BCR, had one of the worst clinical outcomes. A different approach, but also IGHV-determined, led to the categorization of around 30% of CLL cases into different prognostically important subsets, each with highly homogeneous biological features, clinical presentation and outcome. This categorization is based on the observation that, among mutated and unmutated cases, stereotyped BCRs carrying closely homologous heavy chain complementarity determining region 3 (H-CDR3) sequences exist. Following this approach, the CLL cases characterized by the mutated IGHV3-21 could be assigned to the so-called Subset #2 (Stamatopoulos K, Belessi C, Moreno C, et al. Over 20% of patients with chronic lymphocytic leukemia carry stereotyped receptors: pathogenic implications and clinical correlations. Blood. 2007; 109 (1): 259-270; Agathangelidis A., et al. Stereotyped B-cell receptors in one-third of chronic lymphocytic leukemia: A molecular classification with implications for targeted therapies. Blood. 2012; 119:4467-4475).

Notably, the IGHV3-21 usage according to subset #2 has always been observed in association with the expression of an immunoglobulin lambda variable 3-21 chain along with an acquired substitution of glycine with arginine at amino acid position 110 (IGLV3-21R¹¹⁰) in the light chain. Causative for the arginine 110 (R110) of IGLV3-21^{R110} is a single G>C substitution on the splice site between the immunoglobulin lambda J and constant genes. The presence of R110 together with germline encoded lysine 16 (K16) in one BCR, and aspartates (D) 50 and 52 in a tyrosine-aspartate-serine-aspartate (YDSD) motif of a neighbour BCR, has been identified to enable BCR-BCR interactions, thus triggering cell-autonomous signalling (FIGS. 13 and 14; Minici, C. et al., Distinct homotypic B-cell receptor interactions shape the outcome of chronic lymphocytic leukemia, Nature Comm. 2017; 8:15746).

In the course of epigenetic, genomic, and transcriptomic characterization of large cohorts of CLL patients focusing on the BCR light chain, it became clear that around 60% IGLV3-21^{R110} cases carried non-stereotyped BCR, emphasizing that subset #2 is just a minor subgroup of CLL characterized by IGLV3-21^{R110} (Stamatopoulos B, Smith T, Crompot E, et al. The Light Chain IgLV3-21 Defines a New Poor Prognostic Subgroup in Chronic Lymphocytic Leukemia: Results of a Multicenter Study. Clin Cancer Res. 2018; 24 (20): 5048-5057. Nadeu F, Royo R, Clot G, et al. IGLV3-21^{R110} identifies an aggressive biological subtype of chronic lymphocytic leukemia with intermediate epigenetics. Blood. 2021; 137 (21): 2935-2946).

Of the 4 alleles of the IGLV3-21 gene which have been identified in humans, the alleles IGLV3-21*01 (IMGT/LIGM-DB accession No. X71966) and IGLV3-21*04 (IMGT/LIGM-DB accession No. AC279208) encode for the prerequisite K16 and D50 and D52, with the last two incorporated into a motif that, in most cases studied, included a tyrosine at position 49 and a serine at position 51 of IGLV3-21^{R110} (for an exemplarily IGLV3-21R110 see FIG. 14). However, functionally equivalent variations in this motif have also been observed in IGLV3-21^{R110} CLL patients such as the replacement of the tyrosine with a phenylalanine or the serine with a threonine (Nadeu et al. 2021, s.a.). Interestingly, the alleles IGLV3-21*01 and IGLV3-21*04 are strikingly underrepresented in B-cells of healthy donors, whereas all IGLV3-21 genes in patients studied by different groups could be assigned to allele IGLV3-21*01 or allele IGLV3-21*04 suggesting that these alleles might be mechanistically required for the development of the IGLV3-21R110 associated CLL.

The IGLV3-21^{R110} CLL subgroup, for which the name subset #2L has also been proposed, is associated with a very aggressive disease course. Indeed, the poor outcome of the IGLV3-21^{R110} CLL cases is independent of IGHV mutational status or the nature of the heavy chain. Since IGHV3-21^{R110} is found in mutated CLL such as subset #2 (see above) as well as in association with different heavy chains such as IGHV1-18, IGHV3-53 or IGHV3-64 (Nadeu et al. 2021, supra), the IGLV3-21^{R110} defines a group of CLL that is neither limited to the conventional subset classification based on empirically defined epigenetic stereotypes nor to the IGHV-mutational status. Moreover, the essential role of the R110 as a CLL driver mutation has been confirmed by site-specific mutagenesis experiments, which revealed that reversion of IGLV3-21^{R110} into IGLV3-21G110 resulted in abrogation of autonomous signalling capacity of the BCR (Stamatopoulos B, Smith T, Crompot E, et al. 2018, s.a.).

Studies correlating time-to-first treatment (TTFT) and overall survival (OS) with the presence of IGLV3-21R¹¹⁰-carrying BCR showed significant shorter values for patients expressing IGLV3-21^{R110} compared to patients with non-IGLV3-21^{R110} CLLs emphasizing a rapid need for therapy for IGLV3-21^{R110}-positive patients (Nadeu F et al. 2021; s.a.).

However, since CARs targeting common B-cell antigens like CD19 do not discriminate between healthy and malignant B cells, patients are experiencing lasting B cell depletion as long as anti-CD19 CAR T cells persist. Due to the severe hematological toxicity resulting in immune suppression, inability to form vaccine responses, a high infection risk and off-target effects to immune system, no CAR has yet been proven to be suitable for the treatment of CLL (see for e.g. Porter D L, Levine B L, Kalos M et al. Chimeric Antigen Receptor-Modified T Cells in Chronic Lymphoid Leukemia; N Engl J Med 2011; 365:725-733).

From that, a specific CAR for the treatment of IGLV3-21^{R110}-positive CLL patients that does not come along with the aforementioned side effects, still remains to be found.

A CAR is an artificially constructed chimeric transmembrane protein or polypeptide. A transmembrane domain anchors the CAR in the cell membrane of an immune cell and connects an extracellular domain comprising an antigen-binding domain to a cytoplasmic domain that provides the effector function to the immune cell. Immune effector cells engineered to express the genes of the CARs redirect the cells to kill tumors that express a surface antigen targeted by the antigen-binding domain of the CAR. Characteristics of CARs include their ability to redirect immune cell specificity and reactivity toward a selected target in a MHC-independent manner by taking advantage of the antigen-binding properties of an antibody. The antigen-binding domain is traditionally derived from a monoclonal antibody. WO 2019/008129 discloses a monoclonal antibody that can be used to remove CLL cells from blood samples, as a candidate for a fusion protein with T cell specific activation domains in order to obtain a CAR. However, any therapeutic effect of such CAR is not shown. Moreover, the antibodies of WO 2019/008129 have been expressed as IgG antibodies in a hybridoma cell line departing from a murine host. The potential problem with the use of murine monoclonal antibodies as basis for a CAR is that they can be recognized as foreign and an immune response can be provoked in the patient. This immunogenicity may lead to poor persistence capacities in patients, and reactions like Human Anti-Mouse Antibody (HAMA) response may prohibit the repeated administration of CAR exposing cells.

From the foregoing, a treatment option, particularly a CAR that is selective and non-cross-reactive to healthy B cells, which would allow an improved treatment of CLL in IGLV3-21 R110 positive patients with a reduced risk of toxicity and immunogenic reactions is still unknown but required.

### SUMMARY OF THE INVENTION

The present invention solves the above problem, by providing in a first aspect a novel humanized CAR comprising a humanized IGLV3-21^{R110} binding domain, a transmembrane domain, and a cytoplasmic domain, wherein the humanized IGLV3-21 R110 binding domain comprises a light chain variable region (VL) which comprises a light chain complementarity-determining region 1 (L-CDR1) having an amino acid sequence of SEQ ID NO: 1, a light chain complementarity-determining region 2 (L-CDR2) having an amino acid sequence of SEQ ID NO: 2, and a light chain complementarity-determining region 3 (L-CDR3) having an amino acid sequence of SEQ ID NO: 3 and a heavy chain variable region (VH) which comprises a heavy chain complementarity-determining region 1 (H-CDR1) having an amino acid sequence of SEQ ID NO: 4, a heavy chain complementarity-determining region 2 (H-CDR2) having an amino acid sequence of SEQ ID NO: 5, a heavy chain complementarity-determining region 3 (H-CDR3) having an amino acid sequence of SEQ ID NO: 6.

Such humanized CAR specifically binds to the IGLV3-21^{R110}-harboring BCR and thereby, unlike anti-CD19 CARs, mediates the killing of malignant B-cells without off-target killing of healthy B cells of the CLL patient. While recognizing the presence of the IGLV3-21R¹¹⁰-part of the BCR, this new CAR is also capable of targeting “subset #2 CLL”, which is characterized by BCRs comprising an IGHV3-21/IGLV3-21^{R110}combination, or any other disease or CLL comprising IGLV3-21R110.

In a second aspect, the invention is also related to polynucleotides encoding the humanized CARs of the invention. Thus, the invention also pertains to vectors containing a nucleic acid sequence of the invention in a third aspect.

In a fourth aspect, the invention pertains to a cell expressing a humanized CAR according to the present invention.

In a fifth aspect, the invention pertains to a pharmaceutical composition according to the present invention.

In a sixth aspect, the present invention pertains to the provision of a CAR, a polynucleotide, a vector, a cell, or a pharmaceutical composition according to the present invention for use in treating a disease, particularly in the treatment of CLL in IGLV3-21 R110 positive patients. Likewise, this sixth aspect of the present invention pertains to a method for treating a disease, in particular to a method of treatment of CLL in IGLV3-21^{R110} positive patients, by administering a CAR, a polynucleotide, a vector, a cell, or a pharmaceutical composition according to the present invention to a subject in need thereof.

Pursuant to this sixth aspect said CAR, polynucleotide, vector, cell or pharmaceutical composition may be administered in conjunction with an additional therapeutic agent. Specifically, the therapeutic agent may be a Bruton's Tyrosine Kinase (BTK).

A further aspect provides a kit comprising a CAR, a polynucleotide, a vector, a cell, or a pharmaceutical composition according to the present invention.

### Definitions

Unless defined otherwise, all technical and scientific terms used herein have the meaning commonly understood by one of ordinary skill in the art to which this invention belongs. The following references, however, can provide one of skill in the art to which this invention pertains with a general definition of many of the terms used in this invention, and can be referenced and used so long as such definitions are consistent the meaning commonly understood in the art. Such references include, but are not limited to, Singleton et al, Dictionary of Microbiology and Molecular Biology (2d ed. 1994); The Cambridge Dictionary of Science and Technology (Walker ed., 1988); Hale & Marham, The Harper Collins Dictionary of Biology (1991); and Lackie et al., The Dictionary of Cell & Molecular Biology (3d ed. 1999); and Cellular and Molecular Immunology, Eds. Abbas, Lichtman and Pober, 2nd Edition, W.B. Saunders Company.

Any additional technical resources available to the person of ordinary skill in the art providing definitions of terms used herein having the meaning commonly understood in the art can be consulted. For the purposes of the present invention, the following terms are further defined.

As used herein and in the appended claims, the singular forms “a,” “and,” and “the” include plural reference unless the context clearly dictates otherwise. Thus, for example, reference to “a gene” is a reference to one or more genes and includes equivalents there of known to those skilled in the art, and so forth.

An “autonomously active” BCR is a special type of a permanently active BCR. While the conventional activation is based on an external antigen, the autonomously active BCR results from its interaction with membrane structures on the surface of the same cell. For the clinical picture of CLL, an autonomic activation-triggering interaction between BCRs adjacent to each other on the surface of the same cell could be shown (e.g. M. Dühren-von Minden et. al; Nature 2012).

IGLV3-21^{R110} is the light chain variable region of the BCR that enables BCR-BCR interactions, to induce an autonomously active BCR. Structurally such IGLV3-21^{R110} is characterized by a sequence identity of more than 80% to the sequence as represented by SEQ ID NO: 41, wherein in any case at position 110 of said sequence there is Arginine and not Glycin.

The term “antibody”, as used herein, is intended to refer to immunoglobulin molecules, preferably comprised of four polypeptide chains, two heavy (H) chains and two light (L) chains which are typically inter-connected by disulfide bonds. Each heavy chain is comprised of a heavy chain variable region (abbreviated herein as VH) and a heavy chain constant region. The heavy chain constant region can comprise e.g. three domains CH1, CH2 and CH3. Each light chain is comprised of a light chain variable region (abbreviated herein as VL) and a light chain constant region. The light chain constant region is comprised of one domain (CL). The VH and VL regions can be further subdivided into regions of hypervariability, termed complementarity-determining regions (CDR), interspersed with regions that are more conserved, termed framework regions (FR), which provide a suitable scaffold for the CDRs.

As used herein, the term “Complementarity Determining Regions (CDRs; e.g., CDR1, CDR2, and CDR3) refers to the amino acid residues of a variable domain of a molecule capable of binding an antigen, for example an antibody, antibody-fragment, a single chain variable Fragment (scFv), the presence of which are necessary for antigen binding. Each variable domain typically has three CDR regions identified as CDR1, CDR2 and CDR3. The amino acid sequence boundaries of a given CDR can be readily determined using any of a number of well-known schemes, including those described by Kabat et al. (“Sequences of Proteins of Immunological Interest,” 5th Ed. Public Health Service, National Institutes of Health, Bethesda, MD, 1991; “Kabat” numbering scheme), Chothia and Lesk (J Mol Biol 196:901-917 (1987)), and Lefranc et al. (“IMGT unique numbering for immunoglobulin and T cell receptor variable domains and Ig superfamily V-like domains,” Dev. Comp. Immunol., 27:55-77, 2003; “IMGT” numbering scheme). Each complementarity determining region comprises amino acid residues as defined by IMGT. In some instances, a complementarity determining region can also include amino acids from a CDR region defined according to Kabat and/or a hypervariable loop according to Chothia numbering system.

The term “CAR” or “Chimeric Antigen Receptor”, as used herein, is intended to refer to an artificial transmembrane protein which imposes an antigen-triggered response to an immune cell. The CAR engineered immune cell may be for instance a naïve T cell, a central memory T cell, an effector memory T cell, or a Natural Killer (NK) cell. The CAR typically comprises of an extracellular domain and a cytoplasmic domain linked by a transmembrane domain. The extracellular domain comprises at least an antigen-binding domain.

An “antigen-binding domain” hereby is defined as a polypeptide portion of the CAR which recognizes antigen. Specifically, the antigen-binding domain of the invention is a polypeptide having an antigen binding site which comprises complementarity determining regions (CDRs). The antigen-binding domain may comprise 6 CDRs and have an antigen binding site which is equivalent to the heavy chain variable region (VH) and the light chain variable region (VL) of a classical antibody. The VH of a classical antibody results from the immunoglobulin gene rearrangement of V (variable), D (diversity) and J (joining) region gene segments, whereas the VL results from V and J gene segment rearrangement. Each VH and VL generated this way is typically composed of three CDRs and up to four FRs, arranged from amino terminus to carboxy-terminus e.g. in the following order: FR1, CDR1, FR2, CDR2, FR3, CDR3, FR4. The antigen-binding domain is usually a single-chain variable fragment (scFv) derived from an antibody, but it can be based on other formats which include Fab, Fab′, F(ab′) 2, VH and VL antibody chains (Fv fragments), full length heavy and light chains, and linear antibodies.

An “antigen-binding site” typically is found in one or more hyper variable region(s), e.g., the CDR1,-2, and/or 3 regions; however, the variable “framework” regions can also play an important role in antigen binding, such as by displaying the antigen-binding site in an appropriate manner for it to bind the antigen.

A “humanized” CAR as used herein comprises a humanized antigen-binding domain to reduce immunogenicity. Humanized forms of non-human (e.g. murine) antigen-binding domains are genetically engineered chimeric antigen-binding domains whose variable regions, VL and VH, contain minimal sequence derived from non-human immunoglobulin. A variable region of the antigen-binding domain may be one that is (i) CDR-grafted, wherein the CDRs of the variable region are from a non-human origin, while one or more frameworks of the variable region are of human origin; (ii) where amino acids of the framework regions of a non-human variable region are partially exchanged to human amino acid sequences by genetic engineering or (iii) derived from a non-human source (e.g., a transgenic mouse which bears a heterologous immune system), which variable region is based on a human germline sequence. Thus, a humanized antigen-binding domain may be an antigen-binding domain having some or all CDRs from non-human immunoglobulin and variable region framework sequences of one or more frameworks (FRs) from human origin. Likewise, a humanized VL may have at least one, two or three CDRs from non-human immunoglobulin and variable region framework sequences of one, two, three or four frameworks (FRs) from human origin. Likewise, a humanized VH may have at least one, two or three CDRs from non-human immunoglobulin and variable region framework sequences of one, two, three or four frameworks (FRs) from human origin. In some instances, variable region framework residues of human origin may be replaced by corresponding non-human residues. In some instances, the humanized variable region may comprise individual residues which are found neither in the human origin variable region nor in the non-human immunoglobulin CDRs. If the antigen-binding domain contains at least a portion of an immunoglobulin constant region (Fc), the portion is typically that of a human immunoglobulin.

“Percent (%) sequence identity” with respect to a reference polynucleotide or polypeptide sequence, respectively, is defined as the percentage of nucleic acid or amino acid residues, respectively, in a candidate sequence that are identical with the nucleic acid or amino acid residues, respectively, in the reference polynucleotide or polypeptide sequence, respectively, after aligning the sequences and introducing gaps, if necessary, to achieve the maximum percent sequence identity. Conservative substitutions are not considered as part of the sequence identity. Alignment for purposes of determining percent amino acid sequence identity can be achieved in various ways that are within the skill in the art, for instance, using publicly available computer software such as BLAST (Altschul S F, Gish W, Miller W, Myers E W, Lipman D J. Basic local alignment search tool. J Mol Biol 1990; 215:403-10), BLAST-2, ALIGN, LALIGN, or Megalign (DNASTAR) software, or the immunogenetics Information system® (IMGT®) DomainGapAlign tool (Ehrenmann F, Kaas Q, Lefranc M P. IMGT/3D structure-DB and IMGT/DomainGapAlign: a database and a tool for immunoglobulins or antibodies, T cell receptors, MHC, IgSF and MhcSF. Nucleic Acids Res. 2010; 38, D301-307). Those skilled in the art can determine appropriate parameters for aligning sequences, including any algorithms needed to achieve maximal alignment over the full length of the sequences being compared.

As used herein, a CAR “binds specifically to”, is “specific to/for” or “specifically recognizes” an antigen of interest, e.g. a tumor-associated polypeptide antigen target (here, IGLV3-21^{R110}), is able to discriminate between such antigen and one or more reference antigen(s). In its most general form, “specific binding”. “binds specifically to”, is “specific to/for” or “specifically recognizes” is referring to the ability of the CAR to discriminate between the antigen of interest and an unrelated antigen, as determined, for example, in accordance with one of the following methods. Such methods comprise, but are not limited to Flow cytometry, Westem blots, ELISA-, RIA-, ECL-, IRMA-, immunohistological-tests and peptide scans.

“Binding affinity” or “Affinity” refers to the strength of the sum total of noncovalent interactions between a single binding site of a molecule and its binding partner. Unless indicated otherwise, as used herein, “binding affinity” or “Affinity” refers to intrinsic binding affinity which reflects a 1:1 interaction between members of a binding pair (e.g. an antibody and an antigen). Dissociation rate constant K_{D} are usually calculated based on the ratio of equilibrium association (ka) and dissociation rate (ka) constants. The dissociation constant “K_{D}” is commonly used to describe the affinity between a molecule (such as an antibody) and its binding partner (such as an antigen) i.e. how tightly a ligand binds to a particular protein. Ligand-protein affinities are influenced by non-covalent intermolecular interactions between the two molecules. The term “high affinity” means, that the molecule/antibody binds to IGLV3-21R¹¹⁰-positive CLL BCR with an affinity (K_{D}) of lower than or equal to 10-9M (monovalent affinity). The molecule/antibody may have substantially greater affinity for the target antigen compared to other unrelated molecules. Affinity can be measured by common methods known in the art, e.g. according to Example 1.

A “variant” of a CAR contemplated in the invention is a molecule in which the binding activity of the antigen-binding domain for IGLV3-21^{R110} is maintained.

The term “cancer” refers to the physiological condition or disease in which cells divide without control leading to unregulated cell growth. A “tumor” comprises one or more cancer cells.

### DETAILED DESCRIPTION OF THE INVENTION

### First Aspect—of the Invention—CARs

The humanized CARs of the present invention specifically bind BCRs harboring IGLV3-21 R110 and can deliver a therapeutic benefit to a subject. The CARs and their beneficial properties enabling therapeutic activity are described in more detail hereinafter. According to the first aspect of the present invention, humanized chimeric antigen receptors (CARs) comprising a humanized anti-IGLV3-21^{R110} binding domain, a transmembrane domain, and a cytoplasmic domain, wherein the humanized IGLV3-21^{R110} binding domain comprises a light chain variable region (VL) which comprises a light chain complementarity-determining region 1 (L-CDR1) having an amino acid sequence of SEQ ID NO: 1, a light chain complementarity-determining region 2 (L-CDR2) having an amino acid sequence of SEQ ID NO: 2, and a light chain complementarity-determining region 3 (L-CDR3) having an amino acid sequence of SEQ ID NO: 3 and a heavy chain variable region (VH) which comprises a heavy chain complementarity-determining region 1 (H-CDR1) having an amino acid sequence of SEQ ID NO: 4, a heavy chain complementarity-determining region 2 (H-CDR2) having an amino acid sequence of SEQ ID NO: 5, a heavy chain complementarity-determining region 3 (H-CDR3) having an amino acid sequence of SEQ ID NO: 6 are provided.

Preferred embodiments of the first aspect of the present invention are further characterized in more detail in Table 3 of the Examples.

Therefore, a preferred embodiment of the first aspect of the invention is a CAR characterized by a VL having an amino acid sequence selected from the list consisting of SEQ ID NO: 7, SEQ ID NO: 8, SEQ ID NO: 9, and SEQ ID NO: 10 in any combination with a VH having an amino acid sequence selected from the list of SEQ ID NO: 11 and SEQ ID NO: 12.

Within said preferred embodiment of the first aspect of the present invention, CAR “H1” characterized by a VL having an amino acid sequence of SEQ ID NO: 7 and a VH having an amino acid sequence of SEQ ID NO: 11 is a first more preferred embodiment.

Within said preferred embodiment of the first aspect of the present invention, CAR “H2” characterized by a VL having an amino acid sequence of SEQ ID NO: 7 and a VH having an amino acid sequence of SEQ ID NO: 12 is a second more preferred embodiment.

Within said preferred embodiment of the first aspect of the present invention, CAR “H3” characterized by a VL having an amino acid sequence of SEQ ID NO: 8 and a VH having an amino acid sequence of SEQ ID NO: 11 is a third more preferred embodiment.

Within said preferred embodiment of the first aspect of the present invention, CAR “H4” characterized by a VL having an amino acid sequence of SEQ ID NO: 8 and a VH having an amino acid sequence of SEQ ID NO: 12 is a fourth more preferred embodiment.

Within said preferred embodiment of the first aspect of the present invention, CAR “H5” characterized by a VL having an amino acid sequence of SEQ ID NO: 9 and a VH having an amino acid sequence of SEQ ID NO: 11 is a fifth more preferred embodiment.

Within said preferred embodiment of the first aspect of the present invention, CAR “H6” characterized by a VL having an amino acid sequence of SEQ ID NO: 9 and a VH having an amino acid sequence of SEQ ID NO: 12 is a sixth more preferred embodiment.

Within said preferred embodiment of the first aspect of the present invention, CAR “H7” characterized by a VL having an amino acid sequence of SEQ ID NO: 10 and a VH having an amino acid sequence of SEQ ID NO: 11 is a seventh more preferred embodiment.

Within said preferred embodiment of the first aspect of the present invention, CAR “H8” characterized by a VL having an amino acid sequence of SEQ ID NO: 10 and a VH having an amino acid sequence of SEQ ID NO: 12 is an eight more preferred embodiment.

The most preferred CARs of the aforesaid more preferred embodiments of the first aspect of the present invention are those of the first and second more preferred embodiments (“H1” and “H2”).

CARs of this first aspect of the invention are not limited to the specific peptide sequences provided. Rather, the invention also embodies variants. With reference to the instant disclosure and conventionally available technologies and references, the skilled worker will be able to prepare, test and utilize functional variants of the CARs disclosed herein, while appreciating these variants having the ability to bind to the IGLV3-21^{R110}-BCR and thereby killing the B-cell fall within the scope of the present invention.

A variant can include, for example, a CAR that has at least one altered complementarity determining region (CDR) (hyper-variable) and/or framework (FR) (variable) region/position, vis-a-vis a peptide sequence disclosed herein.

As a matter of example, the skilled worker can use the sequences of the CARs provided herein (e.g. of Table 3) to design variants that are within the scope of the present invention.

Furthermore, variants may be obtained by using one CAR of this first aspect of the invention as starting point for optimization by diversifying one or more amino acid residues in the CAR, preferably amino acid residues in one or more CDRs, and by screening the resulting collection of CAR variants.

Polypeptide variants of the CAR binding domain may be made that conserve the overall molecular structure of a CAR described herein. Given the properties of the individual amino acids, some rational substitutions will be recognized by the skilled worker. Amino acid substitutions, “conservative substitutions,” may be made, for instance, on the basis of similarity in polarity, charge, solubility, hydrophobicity, hydrophilicity, and/or the amphipathic nature of the residues involved.

For example, (a) nonpolar (hydrophobic) amino acids include alanine, leucine, isoleucine, valine, praline, phenylalanine, tryptophane, and methionine; (b) polar neutral amino acids include glycine, serine, threonine, cysteine, tyrosine, asparagine, and glutamine; (c) positively charged (basic) amino acids include arginine, lysine, and histidine; and (d) negatively charged (acidic) amino acids include aspartic acid and glutamic acid. Substitutions typically may be made within groups (a)-(d). In addition, glycine and proline may be substituted for one another based on their ability to disrupt a-helices. Similarly, certain amino acids, such as alanine, cysteine, leucine, methionine, glutamic acid, glutamine, histidine and lysine are more commonly found in a-helices, while valine, isoleucine, phenylalanine, tyrosine, tryptophan and threonine are more commonly found in β-pleated sheets. Glycine, serine, aspartic acid, asparagine, and proline are commonly found in turns. Some preferred substitutions may be made among the following groups: (i) S and T; (ii) P and G; and (iii) A, V, L and I. Given the known genetic code, and recombinant and synthetic DNA techniques, the skilled scientist readily can construct DNAs encoding the conservative amino acid variants.

The CARs pursuant to the first aspect of the present invention may also comprise a VL having an amino acid sequence identity of at least 70%, 75%, 80%, 85%, 90%, 95%, 98% to an amino acid sequence as presented by SEQ ID NO: 7, SEQ ID NO: 8, SEQ ID NO: 9, or SEQ ID NO: 10.

The CARs pursuant to the first aspect of the present invention may also comprise a VH having an amino acid sequence identity of at least 70%, 75%, 80%, 85%, 90%, 95%, 98% to an amino acid sequence as presented by SEQ ID NO: 11 or SEQ ID NO: 12. The humanized anti-IGLV3-21^{R110} binding domain of the CARs pursuant to the first aspect of the invention is preferably a scFv. The order of the VL and VH within the scFv may be from the N-terminus to the C-terminus VL-VH or VH-VL, whereby the order VL-VH is preferred. The VL of the scFv is preferably attached to the VH via a suitable amino acid linker. Preferred scFv of the CARs pursuant to the first aspect of the invention have an amino acid sequence of SEQ ID NO: 13, SEQ ID NO: 14, SEQ ID NO: 15, SEQ ID NO: 16, SEQ ID NO: 17, SEQ ID NO: 18, SEQ ID NO: 19, or SEQ ID NO: 20.

The CARs pursuant to the first aspect of the invention may further comprise a transmembrane domain derived from type-1 membrane-spanning proteins like CD28, CD3zeta, CD4, CD8a, or OX40. Preferably, the transmembrane domain is of human origin. Specifically, the transmembrane domain may be derived from human CD28.

The CARs pursuant to the first aspect of the invention may further comprise a spacer region. Frequently, a spacer is necessary to isolate the antigen-binding domain from the membrane to provide access to the antigen and some flexibility. The spacer may be derived from IgG1 or IgG4 or from the extracellular CD28, CD4, or CD8. Specifically, the spacer may be the CH1-CH2-CH3 constant region of IgG1 or IgG4. More specifically, a more compact spacer may suffice, such as the IgG1 or IgG4 hinge. Preferably the spacer may be of human origin.

Pursuant to the first aspect of the present invention, the cytoplasmic domain may comprise, consist essentially of, or consist of a signalling domain. The signalling domain may comprise one or more tyrosine-based activation motifs, preferably immunoreceptor tyrosine-based activation motifs (ITAMs). An ITAM serves as specific adaptor for downstream signalling proteins upon phosphorylation of the conserved tyrosine residue within this motif. The signalling domain may be derived, for example, but not limited to, from CD3ζ, FcεR1, DAP10 or DAP12. The FcεR1 signalling domain comprises one ITAM, the CD3ζsignalling domain contains three ITAMs and associates with T cell receptors to generate a signal. The signalling domain may be or comprise a T cell signalling domain. Preferably, the signalling domain may be of human origin. Specifically, the signalling domain may be derived from CD3ζ.

The cytoplasmic domain may comprise one or more additional costimulatory domains. Suitable costimulatory domains may include CD2, CD27, CD28, 4-1BB (CD137), CD244, ICOS, or OX40 derived domains, which can be useful alone or in any combination thereof. Accordingly, the cytoplasmic domain may comprise a signalling domain alone, or the signalling domain in combination with one or more costimulatory domains. Specifically, the cytoplasmic domain may comprise a CD37 signalling domain in combination with costimulatory domains CD28 and 4-1BB.

The humanized CARs pursuant to the first aspect of the present invention specifically bind to IGLV3-21^{R110} (SEQ ID NO: 41 and FIG. 14). It could be demonstrated with IGLV3-21R¹¹⁰-positive and IGLV3-21R¹¹⁰-negative cell lines and with primary CLL cells that, unlike anti-CD19 CARs, humanized anti-IGLV3-21^{R110} CARs effectively discriminate between the wild-type IGLV3-21G110 variant and the malignant IGLV3-21^{R110} variant (Example 3, FIGS. 3, 4, 6, 9).

Furthermore, as described herein, the CAR T cells based on the humanized anti-IGLV3-21^{R110} CARs have been shown to selectively kill the IGLV3-21^{R110} positive cell lines and primary CLL cells, but not polyclonal non-malignant B cells, which are, however, equally affected by killing by anti-CD19 CAR T cells. Thus resulting in beneficial aspect of the present invention that the humanized anti-IGLV3-21^{R110} CARs specifically and selectively bind to the IGLV3-21^{R110} positive B cells and thereby enable selective killing of malignant cells while sparing healthy human B cells (Example 3; FIG. 7).

Superior killing was observed with anti-IGLV3-21^{R110} CAR based CAR T cells derived from healthy donor T cells (e.g. Example 4 and FIG. 11), and even CLL patients derived CAR T cells revealed an efficient killing capacity despite the well-known functional impairment of T cells in CLL (Example 4 and FIG. 11).

Moreover, the killing of the CLL B cells was accompanied by elevated immune effector cytokine levels (Examples 5 and 6, FIGS. 5, 8,12).

In summary, the excellent suitability of the humanized anti-IGLV3-21^{R110} CARs pursuant to the present invention for precision targeting of IGLV3-21^{R110} positive B cells with CAR T cells has been demonstrated. This precision and their lower immunogenicity render the humanized CARs of the present invention a potential tool in the field of therapeutic treatment of CLL patients, who are characterized by a compromised immune system due to the underlying disease.

Beyond the definition provided for IGLV3-21^{R110} above, said IGLV3-21^{R110} is in a generally preferred embodiment of this first aspect of the invention further characterized by a sequence identity of more than 80% to the sequence as represented by SEQ ID

NO 41, wherein at position 16 of said sequence there is lysine and at positions 50 and 52 there are aspartates. In a generally more preferred embodiment of this first aspect of the invention, at position 49 there is a tyrosine or a phenylalanine and at position 51 there is a serine or a threonine. In a generally further more preferred embodiment of this first aspect of the invention at position 49 there is a tyrosine and at position 51 there is a serine.

### Second Aspect of the Invention-Polynucleotides

The present invention also pertains to polynucleotides that encode a CAR of the first aspect of the invention. Polynucleotides pursuant to the present invention may comprise DNA or RNA.

DNA molecules of the invention are not limited to the sequences disclosed herein, but also include variants thereof. DNA variants within the invention may be described by reference to their physical properties in hybridization. The skilled worker will recognize that DNA can be used to identify its complement and, since DNA is double stranded, its equivalent or homolog, using nucleic acid hybridization techniques. It also will be recognized that hybridization can occur with less than 100% complementarity. However, given appropriate choice of conditions, hybridization techniques can be used to differentiate among DNA sequences based on their structural relatedness to a particular probe. For guidance regarding such conditions see, Sambrook, J., Fritsch, E. F. and Maniatis, T. (1989) Molecular Cloning: A laboratory manual, Cold Spring Harbor Laboratory Press, Cold Spring Harbor, USA and Ausubel et al., 1995 (Ausubel, F. M., Brent, R., Kingston, R. E., Moore, D. D., Sedman, J. G., Smith, J. A., & S1ruhl, K. eds. (1995). Current Protocols in Molecular Biology. New York: John Wiley and Sons).

Structural similarity between two polynucleotide sequences can be expressed as a function of “stringency” of the conditions under which the two sequences will hybridize with one another. As used herein, the term “stringency” refers to the extent that the conditions disfavor hybridization. Stringent conditions strongly disfavor hybridization, and only the most structurally related molecules will hybridize to one another under such conditions. Conversely, non-stringent conditions favor hybridization of molecules displaying a lesser degree of structural relatedness. Hybridization stringency, therefore, directly correlates with the structural relationships of two nucleic acid sequences. The following relationships are useful in correlating hybridization and relatedness (where Tm is the melting temperature of a nucleic acid duplex):

$${T}_{m} = 63.9 + 0.41 ( G + C ) \% . \qquad a$$

$$The {T}_{m} of a duplex DNA decreases by 1 ° C . with every increase \qquad b$$

$$of 1 \% in the number of mismatched base pairs .$$

$$( {T}_{m} ) μ 2 - ( {T}_{m} ) μ 1 = 18.5 {log}_{10} μ 2 / μ 1 \qquad c$$

$$where μ 1 and μ 2 are the ionic strengths of two solutions .$$

Hybridization stringency is a function of many factors, including overall nucleotide concentration, ionic strength, temperature, probe size and the presence of agents which disrupt hydrogen bonding. Factors promoting hybridization include high nucleotide concentrations, high ionic strengths, low temperatures, longer probe size and the absence of agents that disrupt hydrogen bonding. Hybridization typically is performed in two phases: the “binding” phase and the “washing” phase.

Yet another class of polynucleotide variants within the scope of the invention may be described with reference to the product they encode. It is to be understood that a skilled worker may, using routine techniques, make nucleotide substitutions that do not affect the polypeptide sequence encoded by the polynucleotides described herein to reflect the codon usage of any particular host organism in which the products are to be expressed. These functionally equivalent polynucleotides are characterized by the fact that they encode the same peptide sequences found in SEQ ID NOS: 1 to 20 due to the degeneracy of the genetic code.

It is recognized that variants of DNA molecules provided herein can be constructed in several different ways. For example, they may be constructed as completely synthetic DNAs. Methods of efficiently synthesizing oligonucleotides in the range of 20 to about 150 nucleotides are widely available. See Ausubel et al., section 2.11, Supplement 21 (1993). Overlapping oligonucleotides may be synthesized and assembled in a fashion first reported by Khorana et al., J. Mol. Biol. 72:209-217 (1971); see also Ausubel et al., supra, Section 8.2. Synthetic D NAs preferably are designed with convenient restriction sites engineered at the 5′ and 3′ ends of the gene to facilitate cloning into an appropriate vector.

As indicated, a method of generating variants is to start with one of the DNAs disclosed herein and then to conduct site-directed mutagenesis. See Ausubel et al., supra, chapter 8, Supplement 37 (1997). In a typical method, a target DNA is cloned into a single-stranded DNA bacteriophage vehicle. Single-stranded DNA is isolated and hybridized with an oligonucleotide containing the desired nucleotide alteration(s). The complementary strand is synthesized and the double stranded phage is introduced into a host. Some of the resulting progeny will contain the desired mutant, which can be confirmed using DNA sequencing. In addition, various methods are available that increase the probability that the progeny phage will be the desired mutant. These methods are well known to those in the field and kits are commercially available for generating such mutants.

As with the first aspect of the present invention, there are also corresponding preferred embodiments of this second aspect of the invention.

In a first preferred embodiment of this second aspect of the present invention, this invention relates to DNA molecules which encode a CAR, comprising sequences as represented by SEQ ID NO: 21 (for L-CDR1), SEQ ID NO: 22 (for L-CDR2), SEQ ID NO: 23 (for L-CDR3), SEQ ID NO: 24 (for H-CDR1), SEQ ID NO: 25 (for H-CDR2), and SEQ ID NO: 26 (for H-CDR3).

In a second preferred embodiment of this second aspect of the present invention, this invention relates to DNA molecules which encode a CAR, comprising VL sequences as represented by any sequence selected from the list consisting of SEQ ID NO: 27, SEQ ID NO: 28, SEQ ID NO: 29, and SEQ ID NO: 30.

In a third preferred embodiment of this second aspect of the present invention, this invention relates to DNA molecules which encode a CAR, comprising VH sequences as represented by any sequence selected from the list consisting of SEQ ID NO: 31 and SEQ ID NO: 32.

In a forth preferred embodiment of this second aspect of the present invention, this invention relates to DNA molecules which encode a CAR, comprising scFv sequences as represented by any sequence selected from the list consisting of SEQ ID NO: 33, SEQ ID NO: 34, SEQ ID NO: 35, SEQ ID NO: 36, SEQ ID NO: 37, SEQ ID NO: 38, SEQ ID NO: 39, and SEQ ID NO: 40.

### Third Aspect—Vectors

The fifth aspect of the present invention pertains to a vector which comprises a polynucleotide according to the second aspect of the present invention.

Such vector may be used to introduce the polynucleotide into a host cell so that it expresses a CAR according the first aspect of the present invention.

The vector may be a plasmid or a viral vector, preferably a lentiviral or a retroviral vector.

The methods for packaging of polynucleotides into plasmids or viral vectors are known in the art.

The vector may be used to transduce or transfect a cell.

In addition to the gene of interest, the vector may also encode a reporter gene that allows identification, detection and/or selection of the expressed gene of interest in a given host cell using molecular biology and immunology methods known in the art.

Such reporter gene may be a truncated epidermal growth factor receptor (EGFRt) for antibody staining and sorting of CAR-expressing cells in flow cytometry. For co-expression of truncated EGFR, the vector may further encode a viral self-cleaving peptide, e.g. T2A.

### Fourth Aspect-Cells

The fourth aspect of the present invention pertains to a cell expressing a CAR according to the first aspect of the present invention. The cell may be an immune effector cell, such as a T cell, for instance, a naïve T cell, a central memory T cell, an effector memory T cell, a Natural Killer (NK) cell, or a cytokine induced killer cell. Preferably, the cell may be a human T cell.

This fourth aspect also pertains to an in vitro method for making a cell expressing a CAR comprising introducing into an immune cell according to the fourth aspect of the present invention a polynucleotide pursuant to the second aspect of the present invention or a vector according to the third aspect of the present invention under suitable conditions.

### Fifth Aspect—Pharmaceutical Composition

The fifth aspect of the present invention pertains to a pharmaceutical composition comprising a CAR of the first aspect of the present invention, a polynucleotide of the second aspect of the present invention, a vector of the third aspect of the present invention, or a cell of the forth aspect of the present invention and a pharmaceutically-acceptable carrier. Suitable pharmaceutically acceptable carrier may be for instance vehicles, excipients, diluents, and adjuvants. Acceptable carriers are nontoxic to recipients and include, but are not limited to stabilizing agents, buffers such as phosphate, citrate or other organic acids, saline, buffered saline, low molecular weight polypeptides, proteins, such as serum albumin, any sterile, biocompatible pharmaceutical carrier, dextrose, and water. Optionally, the pharmaceutical composition may contain further pharmaceutically active polypeptides and/or compounds.

The pharmaceutical compositions pursuant to the fifth aspect of the present invention may be manufactured in a manner that is known in the art, e.g., by means of conventional mixing, dissolving, emulsifying, encapsulating, entrapping or lyophilizing processes.

After pharmaceutical compositions comprising a CAR of the first aspect of the present invention, a polynucleotide of the second aspect of the present invention, a vector of the third aspect of the present invention, or a cell of the forth aspect of the present invention formulated in an acceptable carrier have been prepared, they can be placed in an appropriate container and labeled for treatment of an indicated condition.

Such administration is usually accomplished parenterally. Methods of parenteral delivery include topical, intra-arterial (directly to the tumor), intramuscular, subcutaneous, intramedullary, intrathecal, intraventricular, intravenous, intraperitoneal, or intranasal administration. The preferred routes of administration are intravenous and intra-arterial (directly to the tumor).

Pharmaceutical compositions for parenteral administration include aqueous solutions of active compounds. For injection, the pharmaceutical compositions of the invention may be formulated in aqueous solutions, preferably in physiologically compatible buffers such as Hank's solution, Ringer's solution, or physiologically buffered saline. Aqueous injection suspensions may contain substances that increase viscosity of the suspension, such as sodium carboxymethyl cellulose, sorbitol, or dextran. Additionally, suspensions of the active compounds may be prepared as appropriate oily injection suspensions.

Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions. For topical or nasal administration, penetrants appropriate to the particular barrier to be permeated are used in the formulation. Such penetrants are generally known in the art.

### Sixth Aspect—Medical Use

The sixth aspect of the present invention relates to the CARs of the first aspect of the present invention, the polynucleotides of the second aspect of the present invention, the vectors of the third aspect of the present invention, the cells of the forth aspect of the present invention, or the pharmaceutical compositions of the fifth aspect of the present invention for use in treating a disease. In particular, the sixth aspect of the present invention pertains to a cell of the forth aspect of the present invention for use in treating a disease.

Likewise this sixth aspect of the present invention relates to a method for treating a disease comprising the step of administering a CAR of the first aspect of the present invention, a polynucleotide of the second aspect of the present invention, a vector of the third aspect of the present invention, a cell of the forth aspect of the present invention, or the pharmaceutical composition of the fifth aspect of the present invention to a subject in need thereof. In particular, the methods pursuant the sixth aspect of the present invention may comprise administering a cell of the forth aspect of the present invention to a subject.

The method pursuant to the sixth aspect of the invention may comprise the steps of:
i) providing a population of immune cells,
ii) introducing into the immune cells a polynucleotide according to the second aspect of the invention or a vector according to the third aspect of the invention,
iii) culturing the immune cells under conditions allowing for expression of the CAR, and administering the cell from (iii) to a subject. Suitably, the cell may be autologous or allogeneic.

Pursuant to this sixth aspect of the present invention, the disease may be chronic lymphocytic leukemia (CLL). Specifically, the disease to be treated is a clinically manifest CLL, which is caused by aberrant proliferation of B-cells that have an autonomously active BCR. More specifically, the disease is a clinically manifest CLL which is characterized by an aberrant proliferation of B-cells that have an autonomously active BCR harboring IGLV3-21 R110.

As discussed in connection with the first aspect of the present invention, humanized anti-IGLV3-21^{R110} CARs have been shown to specifically and selectively mediate cytotoxic killing of malignant B cells expressing IGLV3-21^{R110}-positive BCR by CAR T cells, thus demonstrating the suitability of the humanized anti-IGLV3-21^{R110} CARS pursuant to the present invention for precision treatment of CLL with CAR T cells. Pursuant to the sixth aspect of the present invention, the CAR according to the first aspect of the invention, the polypeptide according to the second aspect of the invention, the vector according to the third aspect of the invention, the cell according to the fourth aspect of the invention, or the pharmaceutical composition according to the fifth aspect of the invention may also be administered in combination with an additional therapeutic agent to a subject. Therefore, the CAR, the polypeptide, the vector, the cell or the pharmaceutical composition may be administered as the sole pharmaceutical agent or in combination with one or more additional therapeutic agents where the combination causes no unacceptable adverse effects. This combination therapy includes administration of a single pharmaceutical formulation which contains the CAR, the polypeptide, the vector, the cell, or the pharmaceutical composition and one or more additional therapeutic agents, as well as administration of a CAR, a polypeptide, a vector, a cell, or a pharmaceutical composition of the present invention and each additional therapeutic agent in its own separate pharmaceutical formulation.

Where separate formulations are used, treatment pursuant to the sixth aspect of this invention with one or more additional therapeutic agents may be at essentially the same time (e.g., concurrently) or at separately staggered times (e.g., sequentially). In particular, the treatment pursuant to the present invention may be performed in fixed or separate combination with other anti-tumor agents such as alkylating agents, anti-metabolites, plant-derived anti-tumor agents, hormonal therapy agents, topoisomerase inhibitors, camptothecin derivatives, kinase inhibitors, targeted drugs, antibodies, interferons and/or biological response modifiers, anti-angiogenic compounds, and other anti-tumor drugs.

A preferred additional therapeutic agent for co-administration pursuant to the sixth aspect of the present invention is a bruton's tyrosine kinase (BTK) inhibitor. Preferably, the BTK inhibitor may be administered in combination with a cell pursuant to the fourth aspect of the present invention, whereby the cell may be autologous.

Certain bruton's tyrosine kinase (BTK) inhibitors are commercially available. Ibrutinib is an inhibitor of that is known to induce apoptosis in B-cell lymphomas and CLL-cells (Hermann S E, Gordon A L, Hertlein E, et al. Bruton tyrosine kinase represents a promising therapeutic target for treatment of chronic lymphocytic leukemia and is effectively targeted by PCI-32765. Blood. 2011; 117:6287-6296). Further BTK inhibitors include e.g. ACP-196 from Acerta Pharma BV and BGB-3111 from BeiGene, Co., Ltd. . . . Preferably, the additional therapeutic agent is ibrutinib. As has been shown in the examples herein, impaired functionality of CLL patient derived CAR T cells could be fully restored by subtherapeutic ibrutinib doses (Example 4, FIG. 11).

### Further Aspect of the Invention-Kit

The invention further relates to pharmaceutical packs and kits comprising one or more containers filled with one or more of the ingredients of the pharmaceutical compositions pursuant to the fifth aspect of the present invention. Associated with such container(s) can be a notice in the form prescribed by a governmental agency regulating the manufacture, use or sale of pharmaceuticals or biological products, reflecting approval by the agency of the manufacture, use or sale of the product for human administration.

A preferred embodiment of the invention is:

A. Humanized chimeric antigen receptors (CARs) comprising a humanized IGLV3-21 R110 binding domain, a transmembrane domain, and a cytoplasmic domain, wherein the humanized IGLV3-21^{R110} binding domain comprises a light chain variable region (VL) which comprises a light chain complementarity-determining region 1 (L-CDR1) having an amino acid sequence of SEQ ID NO: 1, a light chain complementarity-determining region 2 (L-CDR2) having an amino acid sequence of SEQ ID NO: 2, and a light chain complementarity-determining region 3 (L-CDR3) having an amino acid sequence of SEQ ID NO: 3 and a heavy chain variable region (VH) which comprises a heavy chain complementarity-determining region 1 (H-CDR1) having an amino acid sequence of SEQ ID NO: 4, a heavy chain complementarity-determining region 2 (H-CDR2) having an amino acid sequence of SEQ ID NO: 5, and a heavy chain complementarity-determining region 3 (H-CDR3) having an amino acid sequence of SEQ ID NO: 6.

B. CARs according to embodiment A, characterized by a VL having an amino acid sequence selected from the list consisting of SEQ ID NO: 7, SEQ ID NO: 8, SEQ ID NO: 9, and SEQ ID NO: 10 in any combination with a VH having an amino acid sequence selected from the list of SEQ ID NO: 11 and SEQ ID NO: 12.

C. CARs according to embodiment B, characterized by a VL having an amino acid sequence of SEQ ID NO: 7 and a VH having an amino acid sequence of SEQ ID NO: 11, or a VL having an amino acid sequence of SEQ ID NO: 7 and a VH having an amino acid sequence of SEQ ID NO: 12.

D. CARs according to any of the preceding embodiments, characterized by a VL having an amino acid sequence identity of at least 70%, 75%, 80%, 85%, 90%, 95%, 98% to the amino acid sequence of a VL according to embodiment C and an amino acid sequence identity of at least 70%, 75%, 80%, 85%, 90%, 95%, 98% to the amino acid sequence of a VH according to embodiment C.

E. CARs according to any of the preceding embodiments, wherein the humanized anti-IGLV3-21^{R110} binding domain is a scFv.

F. CARs according to embodiment E, wherein the scFv has an amino acid sequence of SEQ ID NO: 13, SEQ ID NO: 14, SEQ ID NO: 15, SEQ ID NO: 16, SEQ ID NO: 17, SEQ ID NO: 18, SEQ ID NO: 19, or SEQ ID NO: 20.

G. A polynucleotide which encodes a CAR according to any preceding embodiments.

H. A vector which comprises a polynucleotide according to embodiment G.

I. A cell expressing a CAR according to any of embodiments A to F.

J. A cell according to embodiment I, wherein the cell is a T cell.

K. A pharmaceutical composition comprising a CAR according to any of embodiments A to F, a polynucleotide according to embodiment G, a vector according to embodiment H or a cell according to embodiments I or J and a pharmaceutically-acceptable carrier.

L. A CAR according to any of embodiments A to F, a polynucleotide according to embodiment G, a vector according to embodiment H, a cell according to embodiment I or J or a pharmaceutical composition according to embodiment K for use in treating a disease.

M. A method for treating a disease, which comprises the step of administering a CAR according to any of embodiments A to F, a polynucleotide according to embodiment G, a vector according to embodiment H, a cell according to embodiments I or J or a pharmaceutical composition according to embodiment K to a subject in need thereof.

N. A method according to embodiment M, which comprises the steps of
iv) providing a population of immune cells,
v) introducing into the immune cells a polynucleotide according to embodiment G or a vector according to embodiment H,
vi) culturing the immune cells under conditions allowing for expression of the CAR, and
vii) administering the cell from (iii) to a subject.

O. The CAR, the polypeptide, the vector, the cell or the pharmaceutical composition for use according to embodiment L or the method according to embodiments M or N, wherein the disease is chronic lymphocytic leukemia (CLL).

P. The CAR, the polypeptide, the vector, the cell or the pharmaceutical composition for use according to embodiment L or O or the method according to any of embodiments M to O, wherein the CAR, the polypeptide, the vector, the cell or the pharmaceutical composition is to be administered in combination with an additional therapeutic agent.

Q. The CAR, the polypeptide, the vector, the cell or the pharmaceutical composition according to embodiment P, wherein the additional therapeutic agent is a bruton's tyrosine kinase (BTK) inhibitor.

R. A kit comprising a pharmaceutical composition according to embodiment K.

### DESCRIPTION OF THE FIGURES

FIG. 1 shows schematic representations of humanized anti IGLV3-21^{R110} CARs.

FIGS. 2A and 2B show lentiviral vectors pJ2459_h1_R110-1BB and pJ2487_h1_R110-1BB for expression of humanized anti IGLV3-21^{R110} CARs.

FIG. 3 shows representative pictures of Cytotoxicity assays after 24 hours co-incubation pursuant to Example 3. FIGS. 3A and 3B depict assays of anti-IGLV3-21R110 CAR T cells from healthy donors expressing humanised scFv H1 (3A: CARHDh1) or scFv H2 (3B: CARHDh2) and OCI-LY1_wt cells (OCI wt) derived of a patient whose CLL is not characterized by a IGHV3-21^{R110}-BCR (i.e. non-IGLV3-21^{R110} CLL). FIGS. 3D and 3E represent the same assays with a generated IGLV3-21^{R110} expressing cell line OCI-LY1_R110 (OCIR110). As can be seen from the comparison of FIGS. 3A and 3D as well as of FIGS. 3B and 3E, anti-IGLV3-21 R110 CAR T cells from healthy donors with humanised scFv H1 and scFv H2 display selective killing of IGLV3-21^{R110} expressing cell line OCI-LY1_R110, indicated by the large bright clusters. FIGS. 3C and 3F depict the controls with untransduced CAR T cells of a healthy donor.

FIG. 4 presents graphs of CAR T cell killing assays performed according to Example 3. FIG. 4A depicts results from co-incubation of OCI-LY1_wt cells (OCI wt) with different healthy donor derived CAR T cells as indicated in the figure; in FIG. 4B, the same set of assays for OCI-LY1_R110 cells is shown. As can be seen from FIG. 4A, OCI-LY1_wt cells were killed by anti-CD19 CAR T cells (CARHDCD19) but remained unaffected by CAR T cells comprising humanized anti-IGLV3-21^{R110} CAR (CARHDh2). By virtue of comparison of FIGS. 4A and 4B, it becomes readily apparent that unlike anti-CD19 CAR T cells, anti-IGLV3-21^{R110} CAR T cells selectively kill IGLV3-21^{R110}positive cell line OCI-LY1_R110.

FIG. 5 shows graphical representations of cytokine quantification in the 24-hour co-culture supernatants of the killing assays depicted in FIG. 4. As can be seen in FIGS. 5A and 5B, IFN-gamma secretion levels as well as IL-6 secretion levels were elevated in the supernatants that showed cell killing by CAR T cells.

FIG. 6 shows graphical representation of killing calculated from CAR T cell killing assays upon co-incubation for 24 hours according to example 3. FIG. 6A depicts killing activity of primary CLL cells from IGLV3-21^{R110}-negative CLL cases (CLL426 and CLL427) by healthy donor derived anti-IGLV3-21^{R110} CAR T cells (CARHDh2), anti-CD19 CAR T cells (CARHDCD19), and anti-TSHR control CAR T cells (CARHDTSHR) as indicated and as compared to untransduced cells (CARHDUTD). FIG. 6B depicts the same analysis as shown in FIG. 6A for the killing of primary CLL cells from IGLV3-21R¹¹⁰-positive CLL cases (CLL438, CLL442). As shown in the comparison of FIGS. 6A and 6B, CD19-directed CAR T cells killed primary CLL cells from both IGLV3-21^{R110}positive and -negative CLL cases, whereas anti-IGLV3-21^{R110} CAR T cells selectively identified and killed IGLV3-21R¹¹⁰-positive CLL cells.

FIG. 7 presents graphs of CAR T cell killing assays quantifying polyclonal B cell killing mediated by healthy donor-derived CAR T cells as compared to untransduced cells (CARHDUTD). The assays were conducted according to Example 3. As can be seen from the graph, polyclonal healthy B cells were killed by anti-CD19 CAR T cells (CARHDCD19) but remained unaffected by anti-IGLV3-21^{R110} CAR T cells indicating that epitope-specific targeting spares normal B cells.

FIG. 8 shows graphical representations of cytokine quantification in the 24-hour co-culture supernatants of the killing assays depicted in FIGS. 6 and 7. As can be seen in FIGS. 8A and 8B, IFN-gamma secretion levels as well as IL-6 secretion levels were elevated in the supernatants that showed cell killing by CAR T cells.

FIG. 9 shows graphical representation of killing calculated from CAR T cell killing assays upon co-incubation for 24 hours according to example 3. FIG. 9A depicts quantification of OCI-Ly1_wt (OCI wt) killing and FIG. 9B depicts quantification of OCI-Ly1_R110 (OCIR110) killing mediated by CLL patient-derived CAR T cells comprising anti-IGLV3-21 R110 CAR (CARCLLh2) or anti-CD19 CAR (CARCLLCD19) as compared to untransduced cells (CARCLLUTD). The assays were conducted with two CLL patients serving as T cell donors, one with active CLL (CLL433) and one with CLL in remission (CLL453). From the comparison of FIGS. 9A and 9B it becomes clearly evident that patient-derived anti-IGLV3-21^{R110} CAR T cells show selective killing of Oci-Ly1-R110 (OCIR110), while anti-CD19 CAR T cells from the same patients killed irrespectively of the neoepitope.

FIG. 10 shows the determination of subtherapeutic dose of ibrutinib. The graph depicts a titration of ibrutinib to determine toxicity towards OCI-Ly1 cell models upon administration of serial dilutions of ibrutinib (11 steps, ranges 0.01 to 20 μM) to OCI-Ly1 wt or OCI-Ly1_R110 cells for 24 hours. Cell growth in percent was plotted as total numbers of ibrutinib-treated cells relative to the total number of untreated cells. This analysis revealed 0.08 UM ibrutinib as highest dose that does not result in a viability drop of target cells. This dose was thus chosen for further testing of CAR T efficacy.

FIG. 11 presents calculated killing graphs from CAR T cell killing assays according to Example 4 showing the effect of BTK-inhibitor ibrutinib in subtherapeutic dose; all cells and addition of ibrutinib as indicated in the graph. The graph depicts the over time monitoring of exclusive killing of IGLV3-21^{R110} expressing cell line (OCIR110) by anti-IGLV3-21^{R110} CAR T cells derived from a healthy donor (CARHDh2) and anti-IGLV3-21 R110 CAR T cells from the blood of a patient with CLL (CLL425, CARCLLh2). Thereby, superior killing was observed with anti-IGLV3-21^{R110} CAR based CAR T cells derived from healthy donor T cells, and CLL patient derived CAR T cells still showed an efficient killing capacity. Furthermore, polyclonal B cells (BHD) of a healthy donor were not affected by these CAR T cells, demonstrating that the CAR T cells of healthy donors and of CLL patients are capable of selectively killing IGLV3-21^{R110}-positive cells, while sparing healthy B cells. Moreover, as shown by the comparison of the cytotoxicity with and without ibrutinib, killing capacity of the CAR T cells derived from healthy donor is not altered by the presence of ibrutinib at subtherapeutic dose throughout the monitoring period of 16 hours. The killing capacity of the CLL patient derived CAR T cells decreased over time in the absence of ibrutinib, whereas the presence of ibrutinib raised the killing capacity of CLL patient derived cells to the level of healthy donor CAR-T cells throughout the monitoring period.

FIG. 12 shows a graphical representation of cytokine IFN-gamma secretion by CAR T cells in the 16-hour co-culture supernatants of the killing assays depicted in FIG. 11. As can be seen from FIG. 12, elevated IFN g levels were measurable in the respective supernatants that showed cell killing by CAR T cells.

FIG. 13 depicts a schematic representation of BCR-BCR homotypic interaction of IGLV3-21 R110 light chains (as described by Minici et al.). Two neighbouring BCRs are depicted with antigen-binding subunit comprising heavy (HC) and light chains (LC), transmembrane domain™, well as the signaling subunit (SU) composed of disulfide-linked heterodimer of the Igα and Igβ proteins (CD79a/CD79b). Mutated arginine at position 110 (R) of one BCR interacts with a germline-encoded aspartate (D) at position 50 of an adjacent BCR. A further interaction between the two BCRs is mediated by the germline-encoded amino acid residues lysine (K) at position 16 and aspartate (D) at position 52.

FIG. 14 shows schematic representation of IGLV3-21^{R110}. FIG. 14A: exemplarily IGLV3-21 R110 (SEQ ID NO: 41) in one-letter code. Amino acid residues involved in BCR-BCR homotypic interactions according to Minici et al. are marked in bold. FIG. 14B: Line 1: Amino acid position in IGLV3-21^{R110}. Line 2: Amino acid residues involved in BCR-BCR homotypic interactions according to Minici et al. Line 3: The YDSD-motif in IGLV3-21R¹¹⁰. Amino acids are depicted in 3-letter code.

FIG. 15 shows the sequences according to SEQ ID Nos: 1 to 55.

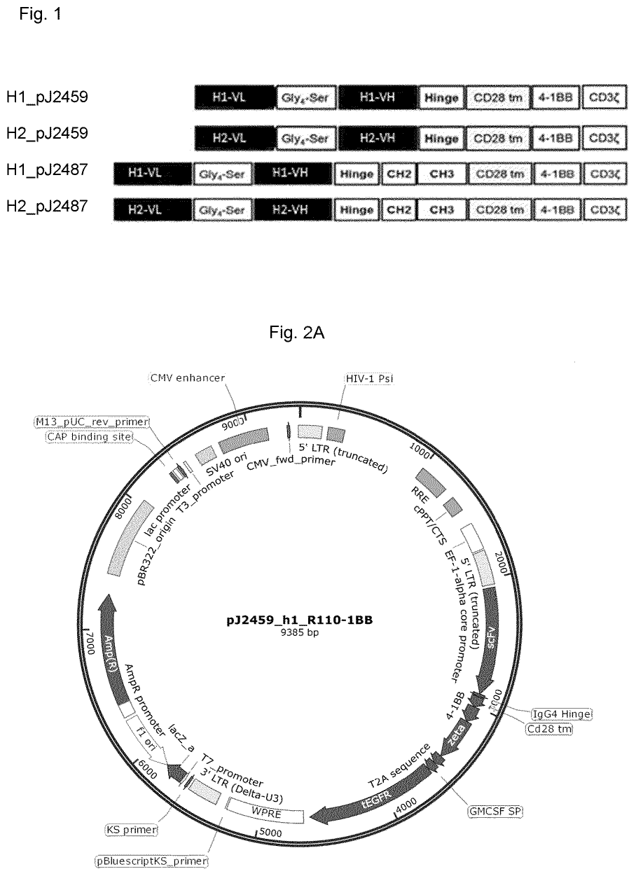

The present invention is further described by the following examples. The examples are provided solely to illustrate the invention by reference to specific embodiments. These exemplifications, while illustrating certain specific aspects of the invention, do not portray the limitations or circumscribe the scope of the disclosed invention.

All examples were carried out using standard techniques, which are well known and routine to those of skill in the art, except where otherwise described in detail. Routine molecular biology techniques of the following examples can be carried out as described in standard laboratory manuals, such as Sambrook et al., 1989 supra.

A preferred embodiment of the invention is:

### EXAMPLES

### Example 1

Design of murine anti-IGLV3-21^{R110} antibody derived humanized scFv and humanized anti-IGLV3-21^{R110} CARS

### 1. Antibodies

1.1. Murine monoclonal antibody mAb01-01

Murine monoclonal antibody mAb01-01 was developed by a combination of immunization of mice with a soluble form of the IGLV3-21^{R110}-harboring BCR (cf. SEQ ID NOS: 42 and 43) and selection of suitable antibodies using a cell system in which the complete and functional BCR was presented membrane bound.

At first, a soluble form of the BCR in the form of an IgG₁ had to be obtained for immunization of mice. Therefore, a DNA segment encoding IGHV3-21 as an exemplary variable heavy chain (VH) and a complete light chain (LC) DNA covering IGLV3-21^{R110} were synthesized by a contract manufacturer using a standard procedure. These were then fused with a murine IgG1 constant segment by polymerase chain reaction (PCR) and cloned into a cytomegalovirus (CMV) vector. A human cellular expression system based on HEK293T cells was used for the expression of such IgG1 (SEQ ID NO: 42 for VH, and SEQ ID NO: 43 for LC) as previously described, e.g. in Rekombinante Antikörper, Lehrbuch und Kompendium für Studium und Praxis, 2. Auflage, Springer Verlag 2019. A polyethyleneimine (PEI) based protocol was used for transfection. After several passages, the supernatant was pooled and the medium contained in the combined cell supernatant was purified using Protein G columns. The purity and quality of the soluble IgG1 was determined by Western blotting.

Thereafter, mice were immunized with the recombinantly produced soluble form of the BCR (cf. SEQ ID NOS: 42 and 43).

Immune cells with the desired specificity could then be obtained from these mice and transformed into hybridoma cells by cell fusion. Monoclonal antibody was produced using the standard procedure in mice and the subsequent generation of hybridoma cells.

The screening for positive clones was not performed by enzyme linked immunosorbent assay (ELISA) as usual. Since the target structure is a membrane-bound receptor, it is of central importance to validate the binding of the potential antibodies in a cellular system, i.e. while largely preserving the cell physiological states native to this cell type. First, groups of pooled supernatants were examined for binding events using fluorescence activated cell sorting (FACS) analysis. For this purpose different BCR variants were expressed on the surface of a triple knockout (TKO) cell line, which cannot express BCR itself.

The starting point for the production of TKO cells is formed by transgenic mice which have a respective knockout for the genes Lambda5, RAG1 or RAG2 and SLP65 (Dühren von Minden et al., 2012, Nature 489, p. 309-313). The combination of the knockouts of RAG2 or RAG1 and Lambda5 leads to a blockade in the transition from the pro-B cell stage to the pre-B cell stage, which is classically characterized by the beginning rearrangement of the VDJ segments of the heavy chain (HC). Therefore they are pro-/pre-B cells. The activity of the BCR can be measured by reconstitution with the inducible SLP65. The production of such mice is known to the expert and belongs to the state of the art. To obtain the cells, the bone marrow of the femur was extracted from the mice after they had been sacrificed. The cells obtained in this way were then cultured under conditions that promote the survival of pro-/pre-B cells (37° C., 7.5% CO2, Iscoves medium, 10% FCS, P/S, murine IL7). After several passages, FACS sorting was carried out for control purposes, the pro-/pre-B cells were sorted and then returned to culture. The markers used for this purpose are known to the specialist.

For reconstitution with a ‘BCR of interest’, the corresponding sequence coding for the VH was fused with a human IgM constant segment by polymerase chain reaction (PCR), and heavy (HC) and light (LC) chains were cloned into respective expression vectors each having a CMV promoter. These were introduced into the packaging cell line (Phoenix cell line) by lipofection. After 36 hours of incubation, the virus supernatant was removed and used for spinfection of the TKO cells. BCR expression was determined using anti-IgM and anti-LC antibodies on FACS. For this purpose, some cells were taken and stained with 5 μl antibody each in a total volume of 100 μl in PBS. Both the work to extract the supernatants and the spinfection of the TKO are widely known procedures and known to experts. Knockout of RAG2 or RAG1 and Lambda5 ensured that only the “BCR of Interest” was expressed on the surface.

In this way, two different BCR-expressing TKO cell lines were generated, one of which expressed the membrane-bound IGHV3-21/IGLV3-21^{R110} BCR. For the generation of the second BCR-expressing TKO cell line, the codon for the arginine at position 110 of the DNA encoding IGLV3-21^{R110} was reverted to the germline sequence by well-known site-directed mutagenesis technique (see, e.g. Sambrook et al., 1989 supra). The resulting TKO cells expressed a BCR containing IGLV3-21 with glycine at amino acid position 110 (IGLV3-21G110). To generate a third control TKO cell line without BCR expression on its surface, spinfection with an empty expression vector was performed. By using an inducible SLP65 to reconstitute the cells, the function of the expressed BCRs could be characterized and the autonomously active state of the IGHV3-21/IGLV3-21^{R110} BCR on the surface could thus be verified before selection. The method of choice here is the measurement of Ca-flux after induction of SLP65 using FACS analysis and the use of a Ca²⁺ dependent dye such as Indo-1. These methods are known to the expert (see M. Duhren-von Minden et. al; Nature 2012).With these cells as “targets”, FACS has now been used to identify an antibody that specifically binds to IGLV3-21^{R110}-harboring BCRs. The first step was to identify the supernatants whose antibodies showed a binding. In this 1st selection round, supernatants of several clones were combined and examined with regard to their binding profile. A positive binding profile is given if a specific binding to the IGHV3-21/IGLV3-21R¹¹⁰-BCR is shown. Groups showing such a profile were isolated, and the binding profile of the individual clones was characterized again during a second selection round. Binding of the monoclonal antibodies was verified using a FACS binding assay using a fluorescently labeled anti-mouse IgG antibody. This approach allowed the isolation of the monoclonal antibody mAb01-01, which demonstrated high specificity and cross-reactivity for human IGLV3-21^{R110}-BCRs without any detectable binding to the wild-type IGLV3-21G110-variant carrying the germline encoded glycine at amino acid position 110.

Antibody mAb01-01 was sequenced. CDRs were identified based on IMGT numbering amino acid annotation. The sequences corresponding to the CDRs of the light chain, L-CDR1, L-CDR2 and L-CDR3 are indicated by SEQ ID NOS: 1, 2 and 3, while the sequences corresponding to the heavy chain CDRs, H-CDR1, H-CDR2 and H-CDR3 are indicated by SEQ ID NOS: 4, 5 and 6. The amino acid sequences of murine variable light and heavy chain were identified as indicated by SEQ ID NO: 44 for the VL and SEQ ID NO: 45 for the VH.

1.2. Humanized anti-IGLV3-21^{R110} antibodies
1.2.1 Humanization of mAB01-01

Humanization of VL and VH was carried out by in silico grafting the murine CDR's into mature human frameworks of variable light and heavy chain regions using standard CDR-grafting technologies by Fusion Antibodies Plc, Belfast, N. Ireland. A number of human framework sequences were identified that were used as “acceptor” frameworks for the mAB01-01 CDR sequences. These acceptor sequences were all derived from mature Human IgG from a human source. As a result, the humanized sequences are expected to be non-immunogenic and retain the canonical structure of the CDR-loops. The human germline gene V-region closest to the murine VH domain was Homo sapiens IGHV4-59. The human germline gene V-region closest to the murine VL domain was Homo sapiens IGKV1-9. New variants were designed based on the variants which showed binding in the previous variants. Germlines that were similar to the parental antibody but had more differentiation in the frameworks were also chosen, CDRs grafted and back mutations introduced to maintain binding. Key residues important for the VL/VH interface and canonical loop structure have been maintained as much as possible in the humanized variants using the CDRx platform (Fusion Antibodies Plc, Belfast, N.Ireland).

The humanized variants were checked to determine whether they had been humanized in accordance with WHO's definition of humanized antibodies (see also World Health Organization (WHO) International Nonproprietary Names (INN) for biological and biotechnological substances (a review) 2014): The variable region of a humanized chain has a V-region amino acid sequence (typically derived from V-region gen segment after somatic hypermutation) which, analysed as a whole, is closer to human V-region germline sequence (i.e., prior to somatic hypermutation) than to those of other species, assessed using the Immunogenetics Information System® (IMGT®) DomainGapAlign tool (Ehrenmann F, Kaas Q and Lefranc M-P 2010 supra).

Table 1 summarizes the best aligned V genes and alleles including the species, the IMGT V gene and allele name, and the Domain label, with the identity percentage and the overlap (number of aligned amino acids assigned to the V-region) between the humanized variants and the IMGT amino acid reference sequence resulting in WHO INN prediction for the humanized variants.

TABLE 1
WHO's assigned antibody INN for the humanized variants

| | | Gene | | | | |
|---|---|---|---|---|---|---|
| | | and | Domain | % | | WHO INN |
| Name | Species | Allele | Label | Identity | Overlap | Designation |
| HC6 | Homo | IGHV4- | VH | 75.3 | 97 | Humanized |
| | sapiens | 4*08 | | | | |
| HC7 | Homo | IGHV4- | VH | 74.2 | 97 | Humanized |
| | sapiens | 4*08 | | | | |
| LC6 | Homo | IGKV1D- | V-Kappa | 83.0 | 94 | Humanized |
| | sapiens | 13*01 | | | | |
| LC7 | Homo | IGKV1- | V-Kappa | 84.0 | 94 | Humanized |
| | sapiens | 9*01 | | | | |
| LC8 | Homo | IGKV3D- | V-Kappa | 85.6 | 90 | Humanized |
| | sapiens | 11*02 | | | | |
| LC9 | Homo | IGKV3- | V-Kappa | 83.3 | 96 | Humanized |
| | sapiens | 20*01 | | | | |

1.2.2 Expression of humanized anti-IGLV3-21^{R110} antibodies

Subsequently, the amino acid sequences of the humanized variants generated by fusion antibodies were converted to nucleotide sequences using Geneiouse software (Geneious Prime 2, Auckland, New Zealand). By fusing VH sequences with a human IgG1 isotype constant domain sequence (SEQ ID NO: 50 for IgG1 constant Nucleotide) and VL sequences with a human IgK isotype constant domain (SEQ ID NO: 51 for IgK constant Nucleotide) by PCR, humanized heavy and light chains were generated, and the antibody gene sequences expressed transiently in Chinese Hamster ovary cells (CHO). The resulting antibody containing cell culture supernatants were clarified by centrifugation and filtration. The humanized antibodies were purified from cell culture supernatant via affinity chromatography. The purity of the antibody was determined to be >95%, as judged by reducing and denaturing Sodium Dodecyl Sulfate Polyacrylamide Gel Electrophoresis (SDS-PAGE). The antibodies were analyzed for protein content and concentration via Seize Exclusion Chromatography (SEC) in PBS-buffer. All steps were performed with state-of the-art equipment and techniques.

This approach resulted in the eight humanized antibodies HC6-LC6, HC7-LC6, HC6-LC7, HC7-LC7, HC6-LC8, HC7-LC8, HC6-LC9, and HC7-LC9.

### 1.2.3 Binding Affinities of the Humanized Anti-IGLV3-21^{r110} Antibodies

After expression of the eight humanized antibodies HC6-LC6, HC7-LC6, HC6-LC7, HC7-LC7, HC6-LC8, HC7-LC8, HC6-LC9, and HC7-LC9, binding tests with a soluble form of the IGLV3-21^{R110} BCR confirmed the high affinity of these antibodies as non-thereof showed values of more than about 3.10-10 M.

In brief, to define the binding affinities of the antibodies to the IGLV3-21^{R110}-harboring B-cell receptor, a soluble recombinant version of the BCR (170.5 kDa; sequence according to SEQ ID NO: 52 for HC and 53 for LC) was produced in 293-HEK cell line as monomeric human IgM by transient expression using a protocol described in section 1.1 of this Example, and binding to immobilized anti-IGLV3-21^{R110} antibodies was monitored by Bio-Layer Interferometry (BLI) on a Fortebio Octet instrument (Satorius). Kinetic assays were performed by first immobilizing the humanized anti IGLV3-21R110 antibodies onto biosensors through an indirect capturing reagent, anti-human lgG Fc antibody. Anti IGLV3-21^{R110} antibodies were loaded at a concentration of 0.01875 μg/ml to generate an anti IGLV3-21^{R110} antibody capture level of between 0.30 and 0.34 nm. A 9 nM BCR-fragment solution in running buffer (PBS, 0.02% Tween20, 0.1% BSA, 0.05% sodium acide) was prepared and serial diluted 1:3 to obtain 7 concentrations from 9 to 0.012 nM (9 nM, 3 nM, 1 nM, 0.333 nM, 0.111 nM, 0.037 nM, and 0.012 nM). The anti IGLV3-21 R110 antibody capture biosensors were then submerged in wells containing the different concentrations of the soluble BCR-fragment for 900 seconds (association stage) followed by a dissociation step of 1200 seconds in running buffer. Steps were performed at a constant shake speed of 1000 rpm. All reagents were used as described by the manufacturer. Sensorgrams were generated after double reference correction (buffer and blank sensors) to compensate for both the natural dissociation of the capture anti IGLV3-21^{R110} antibody and also non-specific binding of the soluble BCR-fragment to the sensor surface. Dissociation rate constants (K_{D}) were calculated based on the ratio of association (ka) and dissociation rate (k_{d}) constants, obtained by fitting sensorgrams with a first order 1:1 binding model using the Fortebio Data Analysis software (Satorius).

TABLE 2
Monovalent K_{D} values of humanized antibody variants as
measured by Fortebio with soluble IGLV3-21^{R110} B-cell receptor
and anti IGLV3-21^{R110} antibody Capture levels

| Humanized | | Capture |
|---|---|---|
| Antibody | K_{D} (M) | level (nm) |
| HC6-LC6 | 2.11E−10 | 0.305 |
| HC7-LC6 | 1.75E−10 | 0.322 |
| HC6-LC7 | 3.47E−10 | 0.337 |
| HC7-LC7 | 2.69E−10 | 0.323 |
| HC6-LC8 | 2.77E−10 | 0.313 |
| HC7-LC8 | 2.49E−10 | 0.330 |
| HC6-LC9 | 2.91E−10 | 0.311 |
| HC7-LC9 | 3.13E−10 | 0.280 |

2. Humanized anti-IGLV3-21^{R110} CARS
2.1. Generation of humanized anti-IGLV3-21^{R110} CARs

Subsequently, a structural model of the humanized single-chain variable fragment (scFv) containing the amino acid sequences of the humanized LC6 and HC7 variants fused with a linker (Li, SEQ ID NO: 46) was predicted (Schrödinger Suit, Schrödinger Inc. NY, USA). The resulting sequence requirements were used as template for the design of the remaining single chain fragments.

The amino acid sequences of the humanized scFv were converted to nucleotide sequences using Geneiouse software (Geneious Prime 2, Auckland, New Zealand) and humanized anti-IGLV3-21^{R110} CARs from scFv were designed using two different CAR constructs:

CAR construct 1 comprises a human IgG4 hinge derived spacer (UniProtKB P01861 IGHG4_HUMAN). Costimulatory domains are derived from CD28 trans-membrane domain (UniProtKB P10747 CD28_HUMAN) and human receptor 4-1BB (UniProtKB Q07011 TNR9_HUMAN). CD3 zeta (UniProtKB P20963 CD3ζ_HUMAN) is used as signaling domain.

CAR construct 2 differs from CAR construct 1 in that it contains an additional spacer sequence derived from CH2-CH3-domain of IgG4 (UniProtKB P01861 IGHG4_HUMAN) 5 between IgG4 hinge and CD28 sequence.

Sequences of the antigen-binding domain of the resulting humanized anti-IGLV3-21R110 CARs are depicted in Table 3.

TABLE 3
humanized anti-IGLV3-21^{R110} CARs, Antigen-binding domain sequences:

| | | | | | | | SEQ ID | SEQ ID | SEQ ID | SEQ ID | SEQ ID | SEQ ID |
|---|---|---|---|---|---|---|---|---|---|---|---|---|
| | SEQ ID | SEQ ID | SEQ ID | SEQ ID | SEQ ID | SEQ ID | NO: | NO: | NO: | NO: | NO: | NO: |
| | NO: | NO: | NO: | NO: | NO: | NO: | VL | VH | scFv | VL | VH | scFv |
| CAR | LCDR1 | LCDR2 | LCDR3 | HCDR1 | HCDR2 | HCDR3 | Protein | Protein | Protein | Nucleotide | Nucleotide | Nucleotide |
| H1 (LC6-Li-HC6) | 1 | 2 | 3 | 4 | 5 | 6 | 7 | 11 | 13 | 27 | 31 | 33 |
| H2 (LC6-Li-HC7) | 1 | 2 | 3 | 4 | 5 | 6 | 7 | 12 | 14 | 27 | 32 | 34 |
| H3 (LC7-Li-HC6) | 1 | 2 | 3 | 4 | 5 | 6 | 8 | 11 | 15 | 28 | 31 | 35 |
| H4 (LC7-Li-HC7) | 1 | 2 | 3 | 4 | 5 | 6 | 8 | 12 | 16 | 28 | 32 | 36 |
| H5 (LC8-Li-HC6) | 1 | 2 | 3 | 4 | 5 | 6 | 9 | 11 | 17 | 29 | 31 | 37 |
| H6 (LC8-Li-HC7) | 1 | 2 | 3 | 4 | 5 | 6 | 9 | 12 | 18 | 29 | 32 | 38 |
| H7 (LC9-Li-HC6) | 1 | 2 | 3 | 4 | 5 | 6 | 10 | 11 | 19 | 30 | 31 | 39 |
| H8 (LC9-Li-HC7) | 1 | 2 | 3 | 4 | 5 | 6 | 10 | 12 | 20 | 30 | 32 | 40 |

### 2.2. T-Cell Epitope Screen

The presentation of peptide sequences in the groove of MHC Class II molecules leads to activation of CD4+ T-cells and an immunogenic response. In order to reduce this response, therapeutic proteins can be designed to avoid the incorporation of T-cell epitopes that can activate T-cells by reducing the affinity of binding to the MHC Class II molecules.

To demonstrate that the antigen-binding domains of the anti-IGLV3-21^{R110} CARS exhibit reduced immunogenicity compared with the murine origin sequences, the humanized VL and VH protein sequences of the binding domains and those of the murine antibody mAB01-01 were screened for MHC Class II binding peptides. Screening was performed using SMM-align as known in the art (Wang P, Sidney J, Kim Y, et al. 2010. Peptide binding predictions for HLA DR, DP and DQ molecules. BMC Bioinformatics. 11:568; Nielsen M, Lundegaard C, Lund O. 2007. Prediction of MHC class II binding affinity using SMM-align, a novel stabilization matrix alignment method. BMC Bioinformatics. 8:238). The following 8 alleles representing over 99% of the world's population were used as the standard allele set for the prediction of MHC Class II epitopes: DRB1*01:01; DRB1*03:01; DRB1*04:01; DRB1*07:01; DRB1*08:02; DRB1*11:01; DRB1*13:02; DRB1*15:01 (Gonzalez-Galarza FF, Christmas S, Middleton D and Jones A R 2011. Allele frequency net: a database and online repository for immune gene frequencies in worldwide populations. Nucleic Acid Research, 39, D913-D919; Greenbaum J, Sidney J, Chung J, et al. 2011 Functional classification of class II human leukocyte antigen (HLA) molecules reveals seven different supertypes and a surprising degree of repertoire sharing across supertypes. Immunogenetics 63 (6): 325-35).

Tables 4 and 5 present the results of VL and VH screen, with high affinity T-cell epitope cores in bold (IC50<50 nM).

Table 4 shows the results of the VL screen. The Human germline sequence IGKV1-9 was also analysed for comparison, and any potential T-cell epitopes present in the germline sequence and matched in the humanized variants, are italicized.

TABLE 4
MHC Class II Epitope Prediction for the Light Chain variants

| SEQ ID | | |
|---|---|---|
| NO: | Name | Amino acid sequence |
| 44 | mAB01- | QIVLTQSPASLSASVGETVTITCRASGNIHSYLAW |
| | 01 VL | YQQKQGKSPQLLVYNAKTLADGVPSRFSGSGSG |
| | | TQYSLKINSLQPEDFGSYYCQHFWNTPPTFGAGT |
| | | KLELK |
| 54 | IGKV1-9 | DIQLTQSPSFLSASVGDRVTITCRASQGISSYLAW |
| | | YQQKPGKAPKLLIYAASTLQSGVPSRFSGSGSGT |
| | | EFTLTISSLQPEDFATYYCQQLNSYP |
| 7 | LC6 | EIVLTQSPSSLSASVGDSVTITCRASGNIHSYLAW |
| | | YQQKPGKAPKLLIYNAKTLADGVPSRFSGSGSGT |
| | | QYTLTISSLQPEDFATYYCQHFWNTPPTFGAGTK |
| | | LELK |
| 8 | LC7 | QIQLTQSPSFLSASVGDSVTITCRASGNIHSYLAW |
| | | YQQKPGKAPQLLIYNAKTLADGVPSRFSGSGSGT |
| | | EYTLTISSLQPEDFATYYCQHFWNTPPTFGAGTK |
| | | LELK |
| 9 | LC8 | EIVLTQSPATLSLSPGERATLSCRASGNIHSYLAW |
| | | YQQKPGQAPRLLIYNAKTLADGIPARFSGSGSGT |
| | | DYTLTISSLEPEDFASYYCQHFWNTPPTFGAGTK |
| | | LELK |
| 10 | LC9 | EIVLTQSPGTLSLSPGERATLSCRASGNIHSYLAW |
| | | YQQKPGQAPRLLIYNAKTLADGIPDRFSGSGSGT |
| | | DYTLTISRLEPEDFAVYYCQHFWNTPPTFGAGTK |
| | | LELK |

Table 5 shows the results of the VH screen. The Human germline sequence IGHV4-59 was also analysed for comparison, and any potential T-cell epitopes present in the germline sequence and matched in the humanized variants, are italicized.

TABLE 5
MHC Class II Epitope Prediction for the Heavy Chain variants

| SEQ ID | | |
|---|---|---|
| NO: | Name | Amino acid sequence |
| 45 | mAB01- | QVQLQQSGPGLVQPSQSLSITCTVSGFSLTSYGIH |
| | 01 VH | WVRQSPGKGLEWLGVIWRGGGTDSNAAFMSRLS |
| | | ITKDNSKSQVFFKMNSLQADDTAIYYCARSRYDEE |
| | | ESMNYWGQGTSVTVSS |
| 55 | IGHV4- | QVQLQESGPGLVKPSETLSLTCTVSGGSISSYYWS |
| | 59 | WIRQPPGKGLEWIGYIYYSGSTNYNPSLKSRVTISV |
| | | DTSKNQFSLKLSSVTAADTAVYYCAR |
| 11 | HC6 | QVQLQESGPGLVKPSETLSLTCTVSGFSLTSYGIH |
| | | WIRQSPGRGLEWIGVIWRGGGTDSNAAFMSRVTI |
| | | SVDTSKSQVSLKLGSVTAADTAIYYCARSRYDEEE |
| | | SMNYWGQGTSVTVSS |
| 12 | HC7 | QVQLQESGPGLVKPSETLSLTCTVSGFSLTSYGIH |
| | | WIRQSPGRGLEWIGVIWRGGGTDSNAAFMSRITIS |
| | | RDTSKTQVSLKLGSVTAADTAIYYCARSRYDEEES |
| | | MNYWGQGTSVTVSS |

As can be readily appreciated from Tables 4 and 5, the murine VH contains a T-cell epitope within the framework 1 region of the sequence, which is not present in the humanized variants (HC6, HC7).

### Example 2

### Generation of CAR-Expressing Primary Human T Cells

To explore the ability of the humanized anti-IGLV3-21^{R110} CARs to redirect immune cell specificity and reactivity toward the IGLV3-21^{R110} positive BCR, T cells that express humanized anti-IGLV3-21^{R110} CARs H1 and H2 were generated by viral vector transduction of T cells using the techniques well known in the art and previously described (e.g., in Yang S, Zhou X, Li R, Fu X, Sun P. Optimized Pei-Based Transfection Method for Transient Transfection and Lentiviral Production. Curr Protoc Chem Biol (2017) 9 (3): 147-57. Epub 2017 Sep. 15. doi: 10.1002/cpch.25). In order to test T cells in different states, a healthy human as well as CLL patients with active CLL (CLL425; CLL433) and with CLL in remission (CLL 453) served as T cell donors.

### Production of CAR Gene Delivery Vehicle Particles (Lentiviral Vector Particles)

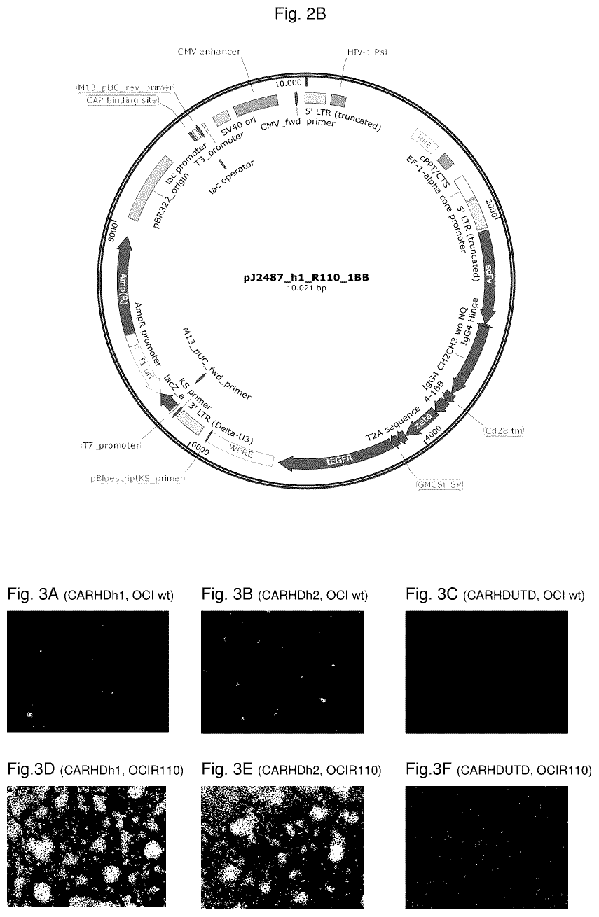

For the expression of scFv in CAR construct 1, vector pJ2459_19loBB (SEQ ID NO: 48) was used, while vector pJ2487_19loBB (SEQ ID NO: 49) was used for the expression of scFv in CAR construct 2. Both vectors for expression of humanized anti-IGLV3-21R110 CAR are shown in FIG. 2.

ScFv sequences were cloned into the two CAR vectors pJ2487_19loBB and pJ2459_19loBB by Nhel (New England Biolabs, Cat. R3131M) and Rsrll (New England Biolabs, Cat. R0501S) restriction sites using In-Fusion® cloning kit (Takara Biosciences, Cat. 639650) according to the manufacturer's instructions.

Lentiviral vector particles were produced in HEK293T cells (DSMZ-German Collection of Microorganisms and Cell Cultures GmbH, Cat. ACC 635). 5×10⁶ HEK293T cells were seeded in a 25 cm² culture flask in DMEM GlutaMAX™ (Thermo Fisher, Cat. 31966021) supplemented with 10% (v/v) FBS (Life Technologies, Cat. 10500064) and 1% (v/v) penicillin-streptomycin (10,000 U/mL) (Thermo Fisher, Cat. 15140122) and incubated at 37° C. in a humidified atmosphere including 5% CO₂. After 18-24h, the medium was replaced with 10 mL fresh medium including 25 μM Chloroquine (Sigma, Cat. C6628). 15 μg of CAR construct vector were mixed with 10 μg of gag/pol plasmid pMDLg/pRRE (Addgene, Cat. 12251), 5 μg of rev plasmid pRSV-Rev (Addgene, Cat. 12253) and 2 μg of envelope plasmid pCMV-VSV-G (Addgene, Cat. 8454) in 500 μL of 250 mM CaCl₂) (Sigma, Cat. C1016). 500 μL of 2× HEPES-buffered saline (Thermo Fisher Cat. 15488749) were added to the DNA mixture, incubated for 20 min at room temperature and added to the cells. The cell medium was collected after 24, 48 and 72 h, filtered through a 0.45 μm syringe filter (Sarstedt, Cat. 83.1826) and stored at −80° C. until transduction.

### T Cell Isolation and Transduction

Peripheral blood mononuclear cells (PBMCs) were isolated from blood of healthy donor or CLL patients collected in K3 Ethylenediaminetetraacetic acid (EDTA)S-Monovetten (EDTA-tubes, Sarstedt A G, Cat. 02.1066.001) EDTA-tubes. Whole blood was layered onto Lymphocyte Separation Medium (Anprotec, Cat. AC-AF-0018) and centrifuged at 500×g for 30 minutes without break. The PBMCs were harvested from the interphase and washed with phosphat buffer saline (PBS). In the case of CLL samples only, PBMCs were depleted of B cells using human B Cell Isolation Kit II (Miltenyi, Cat. 130-091-151) on a Miltenyi autoMACS® instrument according to the manufacturer's instructions. T cells were isolated based on negative selection using Pan T Cell Isolation Kit (Miltenyi, Cat. 130-096-535) on a Miltenyi autoMACS® instrument according to the manufacturer's instructions.

All T cells were expanded at 37° C. in a humidified atmosphere including 5% CO₂ in RPMI 1640 Medium GlutaMAX™ Supplement (Thermo Fisher, Cat. 61870044) supplemented with 10% (v/v) FBS (Life Technologies, Cat. 10500064), 1% (v/v) penicillin-streptomycin (10,000 U/mL) (Thermo Fisher, Cat. 15140122), 50 UM β-mercaptoethanol (Thermo Fisher, Cat. 31350010) and fed 50 U/mL IL-2 (StemCell, Cat. 78036.1) every 48 h (CTL medium). 1 Mio T cells were plated in 2 mL CTL medium per well of a 24-well plate together with washed CD3/CD28 beads (ImmunoCult™ Human CD3/CD28 T Cell Activator, StemCell, Cat. 10971) at a 1:1 bead: T cell ratio and 50 U/mL IL-2 (StemCell, Cat. 78036.1). After 24 h, 1.3 mL medium was removed per well, 5 μg/mL Polybrene (Merck, Cat. TR-1003-G) and lentiviral particles were added and centrifuged for 45 min at 800 g and 32° C. without brake. Cells were incubated for 4 h at 37° C. and fresh CTL medium was added. The next day and then every two days, half of the medium was changed and fresh IL-2 was added. After 6 days, CD3/CD28 beads were removed. Cells were expanded for up to 21 days. Transduction efficiency was checked by flow cytometry on a BD FACSCalibur™ instrument using anti-EGFR-antibody cetuximab (E Cells in the fluorescent channel FL1 above the threshold of unstained controls were considered as successfully transduced. Transduction efficiency above 80% was accepted.

The generated anti-IGLV3-21^{R110} CAR-T cells are depicted in Table 6.

TABLE 6

| CAR-T cell | scFv | T cell origin | vector |
|---|---|---|---|
| CARHDh1 | H1 | healthy donor | pJ2459_19IoBB |
| CARHDh2 | H2 | healthy donor | pJ2459_19IoBB |
| CAR87HDh1 | H1 | healthy donor | pJ2487_19IoBB |
| CAR87HDh2 | H2 | healthy donor | pJ2487_19IoBB |
| CLL425 CARCLLh1 | H1 | CLL patient 425 | pJ2459_19IoBB |
| CLL425 CARCLLh2 | H2 | CLL patient 425 | pJ2459_19IoBB |
| CLL433 CARCLLh1 | H1 | CLL patient 433 | pJ2459_19IoBB |
| CLL433 CARCLLh2 | H2 | CLL patient 433 | pJ2459_19IoBB |
| CLL453 CARCLLh1 | H1 | CLL patient 453 | pJ2459_19IoBB |
| CLL453 CARCLLh2 | H2 | CLL patient 453 | pJ2459_19IoBB |

### Example 3

In-vitro cytotoxicity assay with humanized anti-IGLV3-21^{R110} CAR-T cells 5 A. Provision of target and effector cells

a) Target cells:

OCI-LY1 wt, OCI-LY1 R110, primary CLL PBMCs or healthy B cells were used to test CAR efficacy.

a.1) OCI-LY1 wt cell line (OCI wt)

OCI-LY1 (wild-type) wt cell line, derived from the bone marrow of a 44-year-old man with B-cell non-Hodgkin lymphoma, was purchased from the DSMZ (German Collection of Microorganisms and Cell Cultures GmbH, Cat. ACC722).

a.2) OCI-LY1 R110 cell line (OCIR110)

Neoepitope (i.e. cancer-specific peptide) engineered cell line OCI-LY1 R110 expressing the IGLV3-21^{R110} light chain as part of a BCR with induced R110 point mutation was generated from OCI-LY1 wt cells. First, OCI-LY1 wt cells were cultivated in RPMI 1640 Medium, GlutaMAX™ Supplement (Thermo Fisher, Cat. 61870044) supplemented with 20% (v/v) FBS (Life Technologies, Cat. 10500064) and 1% (v/v) penicillin-streptomycin (10,000 U/mL) (Thermo Fisher, Cat. 15140122) at 37° C. in a humidified atmosphere including 5% CO₂. For ectopic expression of the IGLV3-21^{R110} light chain as a part of a hybrid BCR, the coding sequence (SEQ ID NO: 47) was cloned into the Lentiviral Gene Ontology (LeGO) vector LeGO-iC2-Puro+ (Addgene, Cat. 27345) via AsiSI (New England Biolabs, Cat. R0630S)/EcoRI (New England Biolabs, Cat. R3101L). Lentiviral particles were generated according to the manufacturer's protocol from HEK293T cells (DSMZ-German Collection of Microorganisms and Cell Cultures GmbH, Cat. ACC 635) and the OCI-LY1 wt cells were transduced with the viral particles. As already pointed out in connection with Example 2, both the work to generate the lentiviral particles and the Transduction of the cells are widely known procedures and known to experts (see for example Yang S et al. (2017) supra; Weber K, Bartsch U, Stocking C, Fehse B. A Multicolor Panel of Novel Lentiviral “Gene Ontology” (Lego) Vectors for Functional Gene Analysis. Molecular Therapy (2008) 16 (4): 698-706.). Expression of IGLV3-21^{R110} light chain was confirmed by flow cytometry using APC-anti-IGLV3-21^{R110}-antibody (AVA

LifeScience, Cat. AVA-D01-APC/01P0250). The OCI-LY1 R110 cells were cultivated as described for OCI-LY1 wt.

### a.3) Primary CLL PBMCs

Primary CLL PBMCs were isolated from blood of CLL patients with R110 point mutation (CLL425; CLL438; CLL442) and from CLL patients with wt IGLV3-21G110 light chain suffering from a different kind of CLL (CLL399; CLL426; CLL427) and collected in EDTA-tubes. Whole blood was layered onto Lymphocyte Separation Medium (Anprotec, Cat. AC-AF-0018) and centrifuged at 500×g for 30 minutes without break. The PBMCs were harvested from the interphase and washed with phosphat buffer saline (PBS).

### a.4) Healthy B Cells: Primary Polyclonal B Cells (BHD)

PBMCs were isolated from blood of healthy donors and collected in EDTA-tubes. Whole blood was layered onto Lymphocyte Separation Medium (Anprotec, Cat. AC-AF-0018) and centrifuged at 500×g for 30 minutes without break. The PBMCs were harvested from the interphase and washed with phosphat buffer saline (PBS). Primary polyclonal non-malignant B cells were isolated using human B Cell Isolation Kit II (Miltenyi, Cat. 130-091-151) on a Miltenyi autoMACS® instrument according to the manufacturer's instructions.

b) Effector cells:
b.1) Anti-IGLV3-21^{R110} CAR T cells

CAR T cells from healthy donor, from CLL patients with active CLL (CLL425; CLL433), or with CLL in remission (CLL453) expressing humanized anti-IGLV3-21^{R110} CAR and prepared according to Example 2 were used.

b.2) Healthy Donor and CLL patient untransduced CAR T cells

Untransduced T cells from healthy donor (CARHDUTD, control) and from CLL patients (e.g. CLL433 CARCLLUTD; CLL453 CARCLLUTD) isolated according to Example 2 served to reduce background.

b.3) Anti-human thyrotropin receptor (TSHR) CAR T cells (CARHDTSHR)

CAR T cells from healthy donor directed to human thyrotropin receptor were used as control. For the generation of anti-TSHR CAR T cells, the paratope sequence of a human thyrotropin receptor (TSHR) targeting autoantibody was taken from the literature (Rees Smith, B., Sanders, J., Evans, M., Tagami, T. & Furmaniak, J. TSH receptor-autoantibody interactions. Horm Metab Res 41, 448-455 (2009)), translated into scFv-format, and cloned into the pJ2459 lentivector.

b.4) Anti-CD19 CAR T cells

pJ2459-CD19-targeting CAR as previously published by Hudecek, M., et al. (The nonsignaling extracellular spacer domain of chimeric antigen receptors is decisive for in vivo antitumor activity. Cancer Immunol Res 3, 125-135 (2015)) was used to equip T cells of healthy donor (CARHDCD19) and of CLL patients (e.g. CLL433 CARCLLCD19; CLL453 CARCLLCD19) by lentiviral transduction.

B. Cytotoxicity assay

Target cells seeded at 2×10⁴ cells/well in a 96-well plate were co-incubated with effector cells at effector-to-target (E: T) ratio 5:1 in complete CTL medium consisting of RPMI 1640 Medium GlutaMAX™ Supplement (Thermo Fisher, Cat. 61870044) supplemented with 10% (v/v) FBS (Life Technologies, Cat. 10500064), 1% (v/v) penicillin-streptomycin (10,000 U/mL) (Thermo Fisher, Cat. 15140122), 50 μM β-mercaptoethanol (Thermo Fisher, Cat. 31350010) and 50 U/mL IL-2 (StemCell, Cat. 78036.1). CAR T cell mediated tumor cell killing was assessed using the IncuCyte@ Caspase-3/7 Green Reagent for Apoptosis (Sartorius, Cat. 4440) according to the manufacturer's instructions. Read out of dead (-green-fluorescent) target cells was performed on an

IncuCyte@ S3 (Sartorius) after 2 to 16 hours or 4 to 36 hours. The killing graphs were calculated by the IncuCyte@ software (version 2022A). The total fluorescence per well and timepoint was measured relative to control wells and recorded after a specific point in time or plotted over time. Target cells in combination with untransduced T cells were used as control to set the background. Additional control assays were performed either with only the effector cells or with the target cells only. All values were obtained in triplicates.

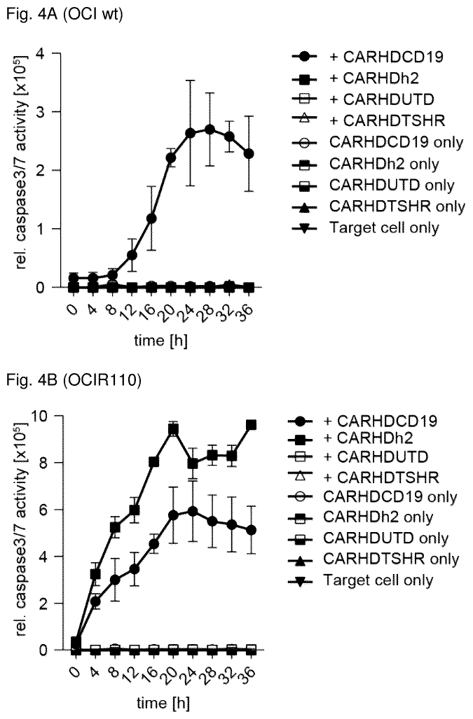

As can be readily appreciated from FIG. 3, which shows representative pictures of the Cytotoxicity assays, and the killing graphs presented in FIGS. 4, 6, 7, and 9, unlike anti-CD19 CAR T cells, CAR T cells with humanized anti-IGLV3-21^{R110} scFv sequence selectively kill IGLV3-21 R110 expressing cell line and primary CLL cells.

The cytotoxicity assays also revealed that the killing capacities of the scFv H1 and scFv H2 were remarkably higher in CAR construct 1 than in CAR construct 2, which may obviously be due to the shorter spacer between the binding domain and the transmembrane domain of CAR construct 1.

Over time monitoring as depicted in FIG. 4 showed exclusive killing of IGLV3-21R110 expressing cell line by anti-IGLV3-21^{R110} CAR T cells derived from healthy human donors. IGLV3-21R¹¹⁰-negative cell line was unaffected by these CAR T cells.

Untransduced control CAR T cells prepared from healthy donor or CLL patients neither killed the IGLV3-21^{R110}-positive nor-negative cell lines (FIGS. 4, 6, and 9). Likewise, both cell lines remained unaffected by healthy donor-derived anti-TSHR CAR T cells (FIGS. 4, 6, 9).

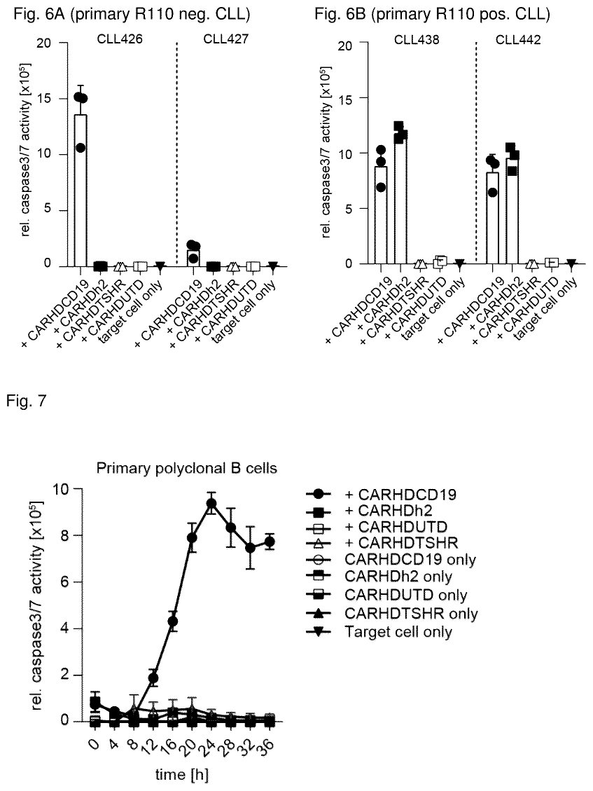

The cytotoxicity assays also showed selective killing of freshly prepared IGLV3-21^{R110}-positive CLL cells by the CAR T cells with humanized anti-IGLV3-21^{R110} scFv sequence derived from healthy human donors (FIG. 6). Isolated primary polyclonal non-malignant B cells remained also unaffected by these cells indicating that epitope-specific targeting spares normal B cells (FIG. 7). In contrast, anti-CD19 CAR T cells equally killed all B cells tested, irrespective of the presence of the IGLV3-21^{R110}-epitope (e.g. FIGS. 6 and 7).

To extend the investigations to more primary CLL target-cells expressing the IGLV3-21^{R110} light chain, additional cytotoxicity assays with defrosted CLL samples were performed. Although the background was generally higher in these experiments due to lower viability of the primary CLL cells, specific killing of IGLV3-21^{R110}-positive CLL cells could be confirmed.

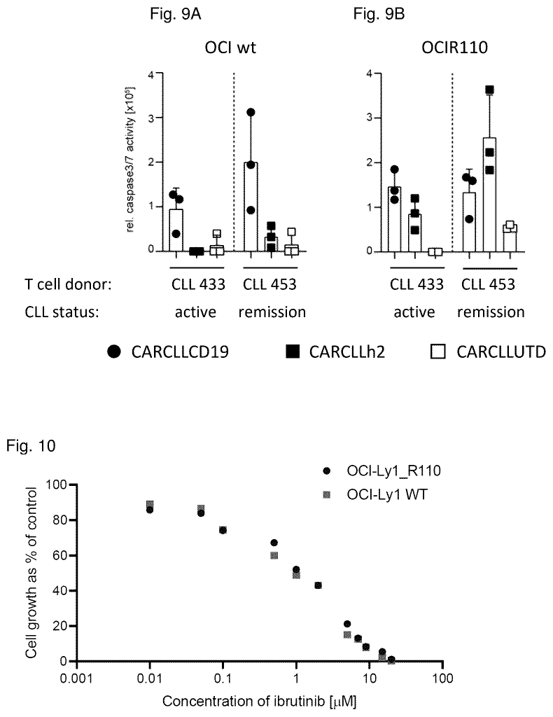

Moreover, CAR T cells from the blood of patients with CLL demonstrated their capacity to selectively kill IGLV3-21^{R110}-positive cells despite the well-recognized functional compromise of T cells in this disease, as can be seen representatively in FIG. 9 using killing graphs generated with CAR T cells from two CLL patients, one with active CLL and one with CLL in remission.

### Example 4

Comparative Cytotoxicity Assay with Humanized Anti-IGLV3-21^{r110} CAR-T Cells in the Presence of BTK-Inhibitor

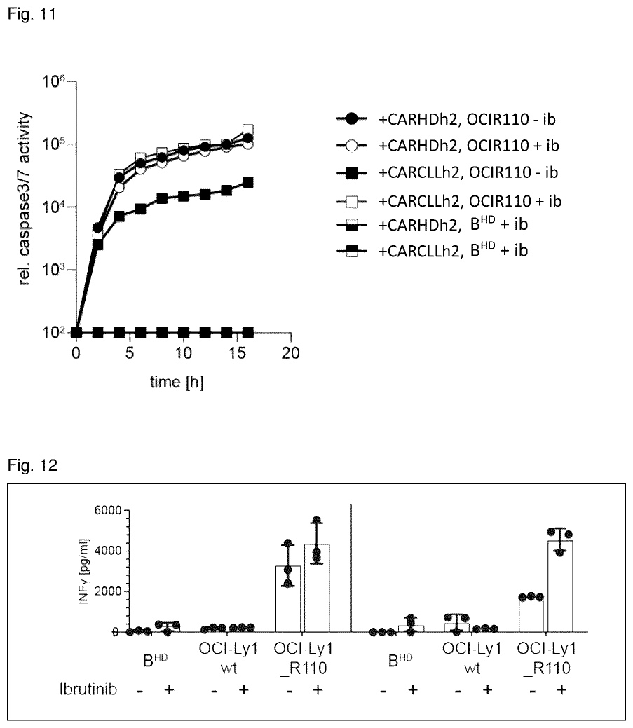

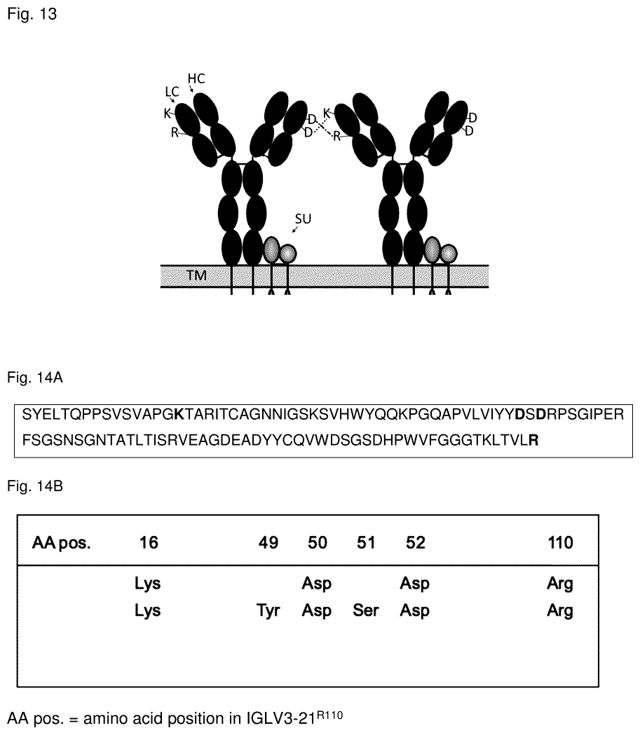

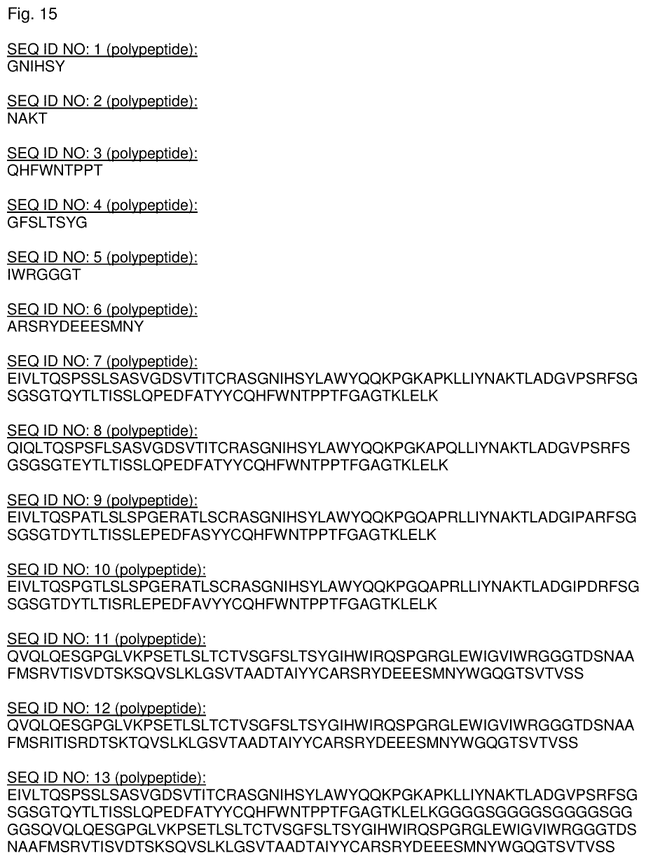

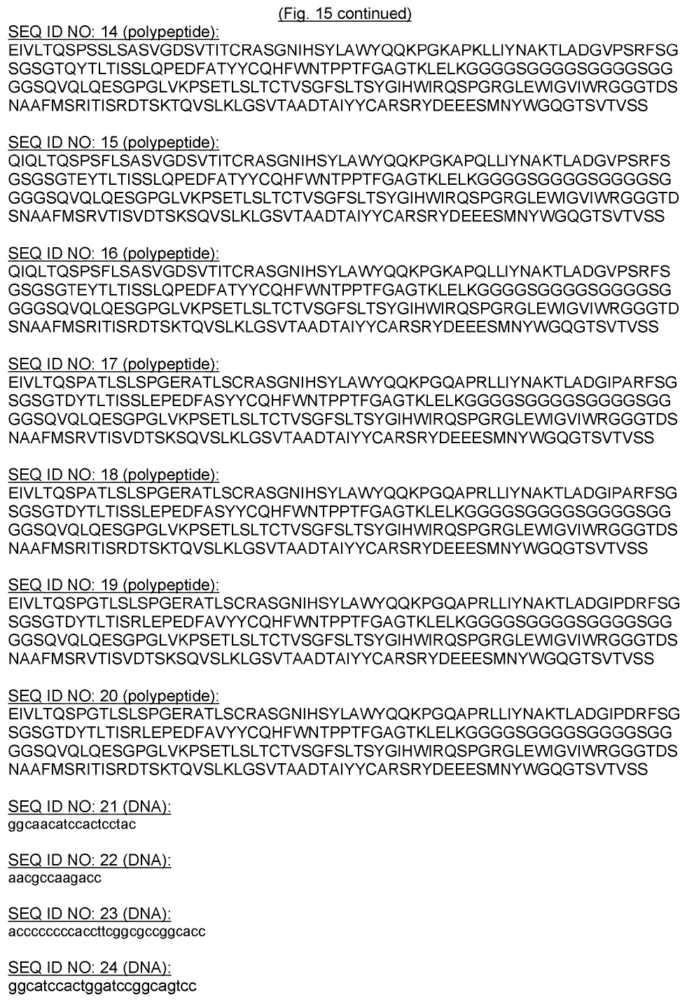

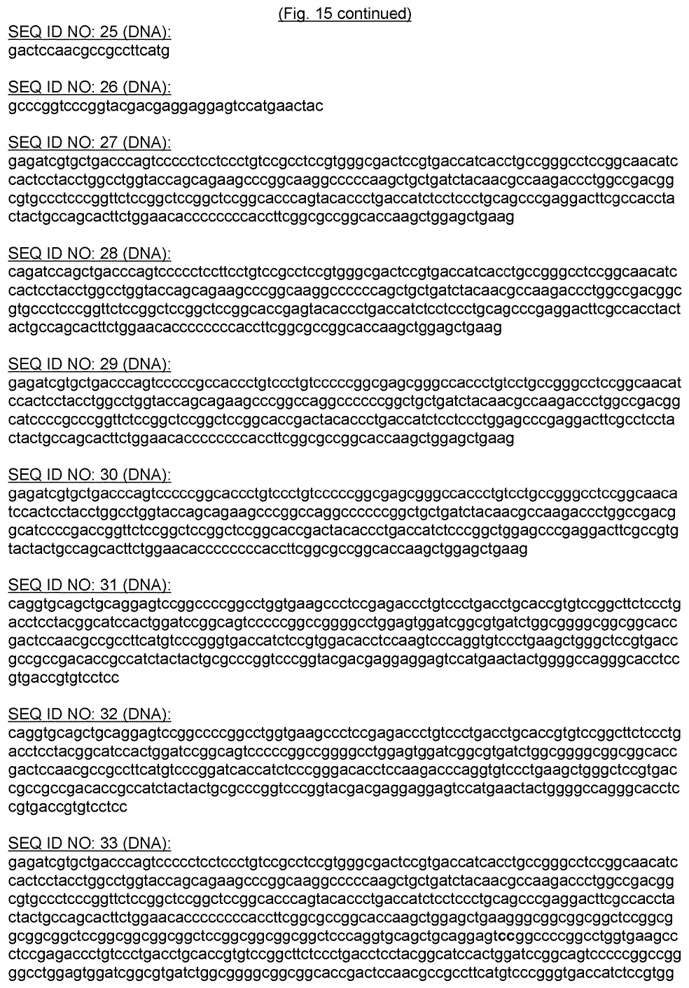

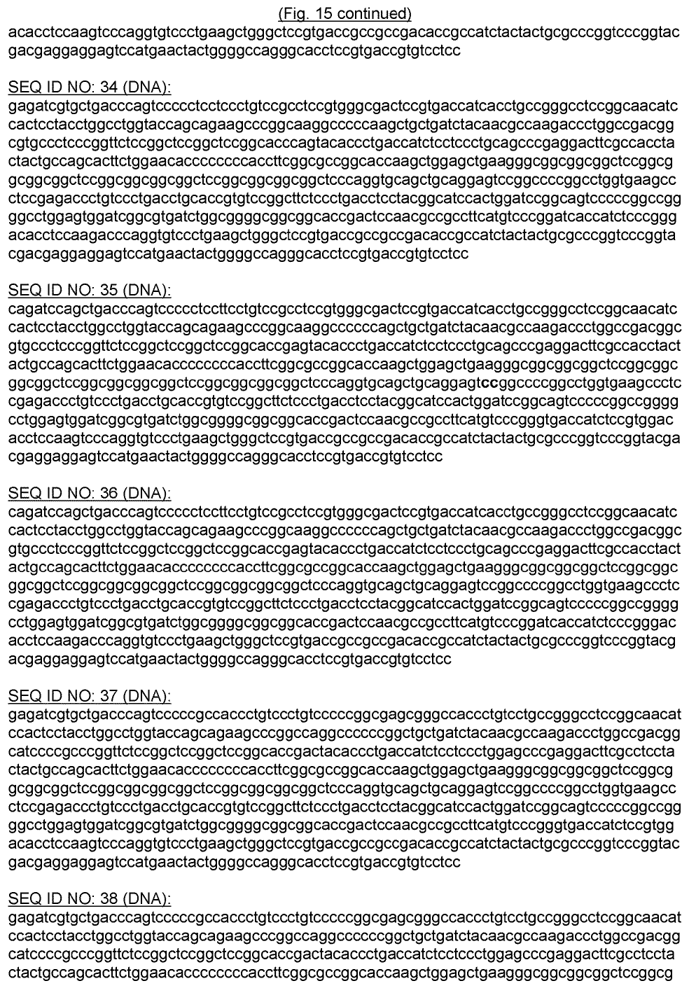

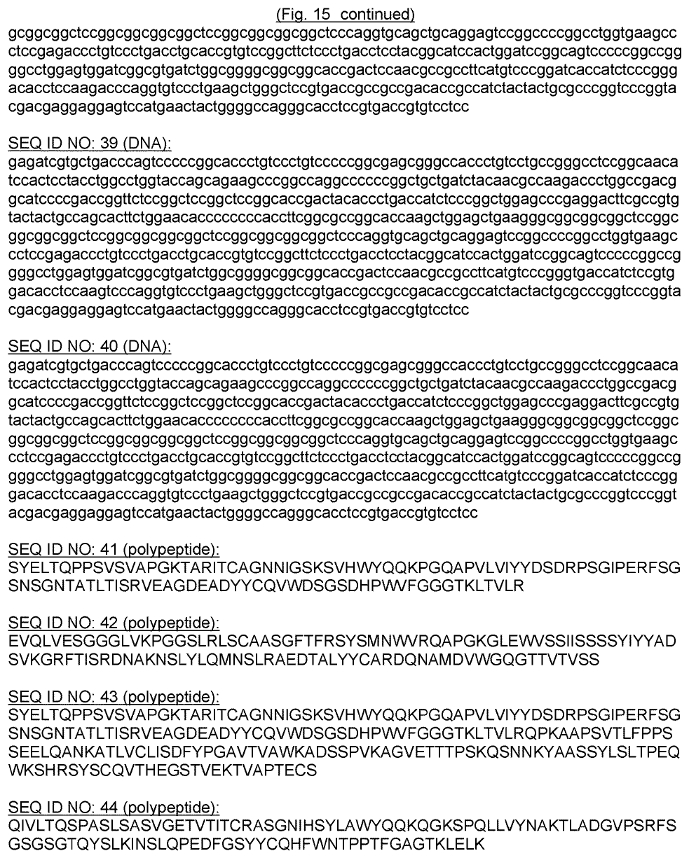

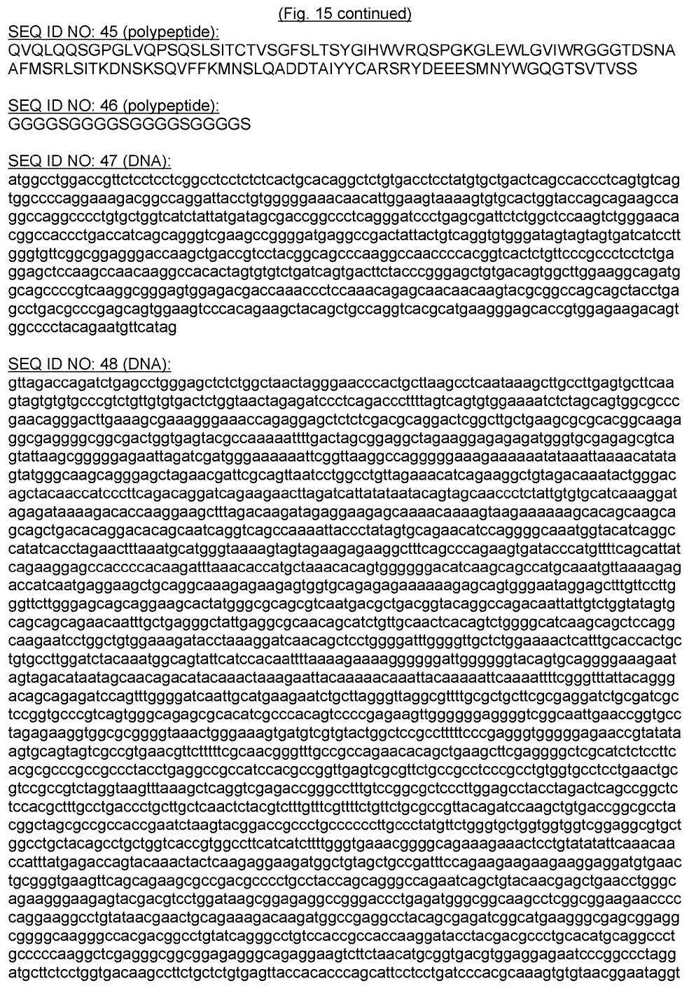

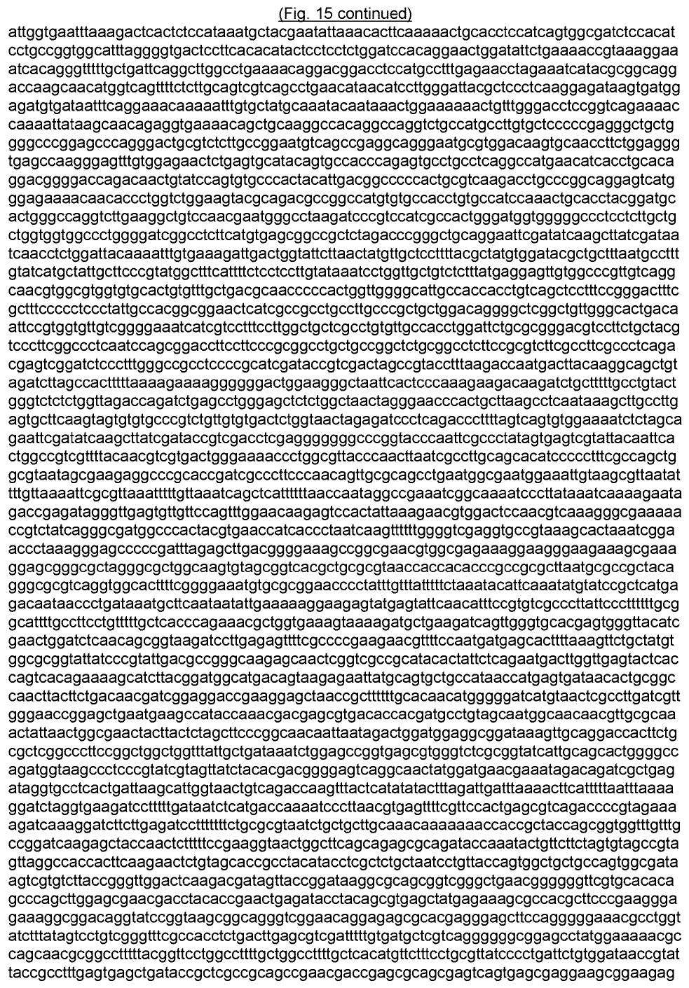

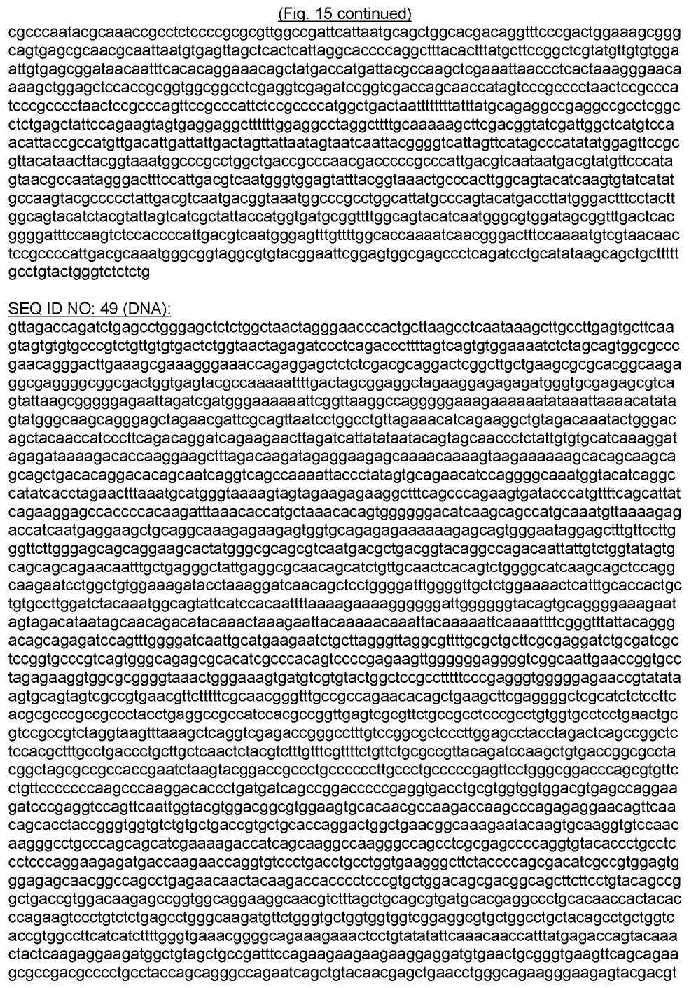

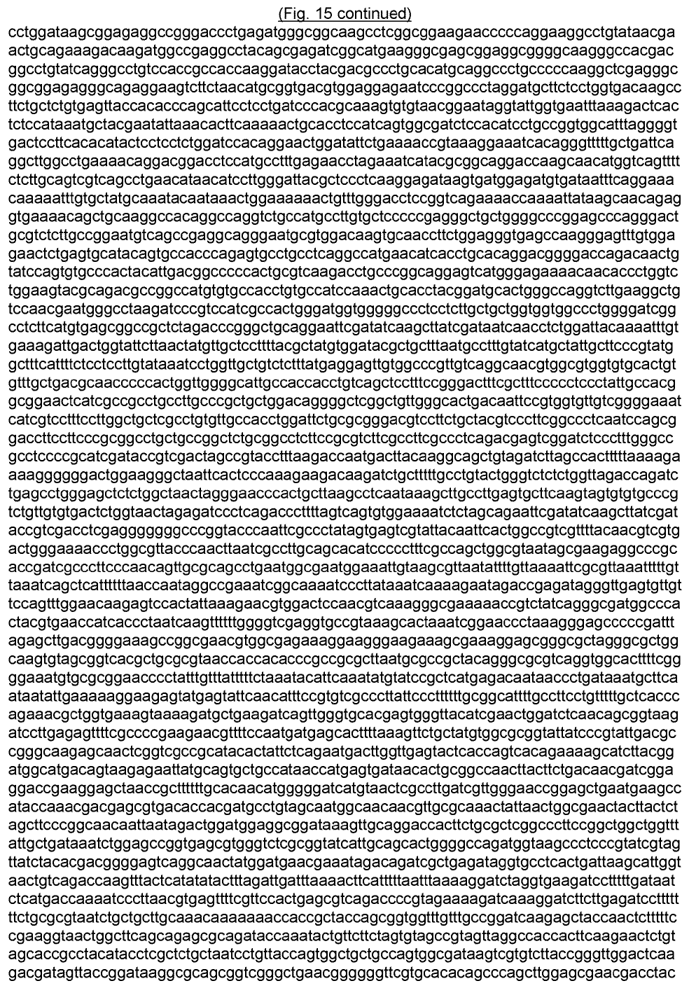

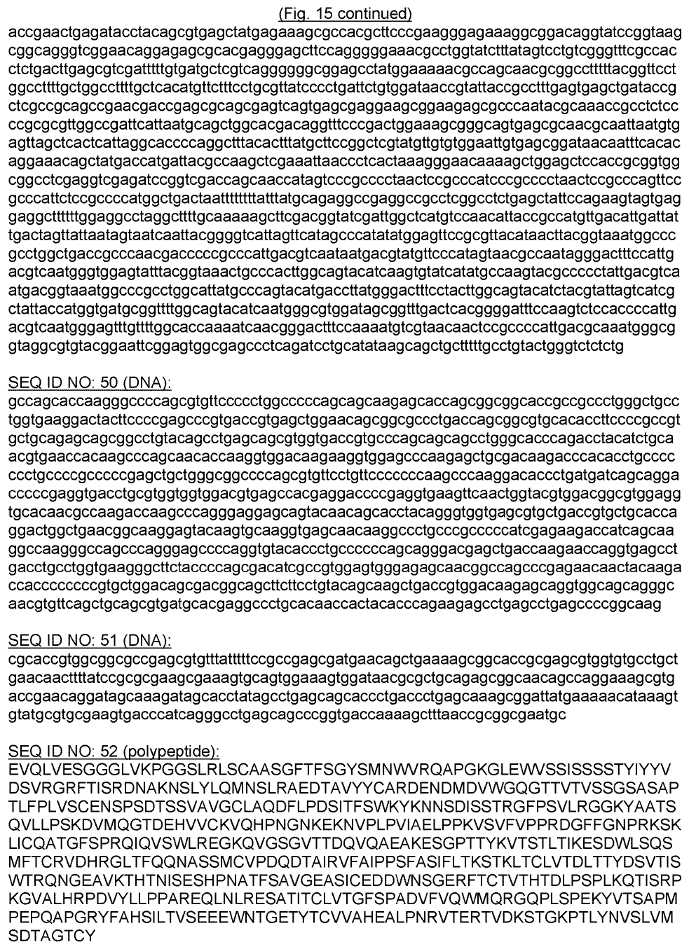

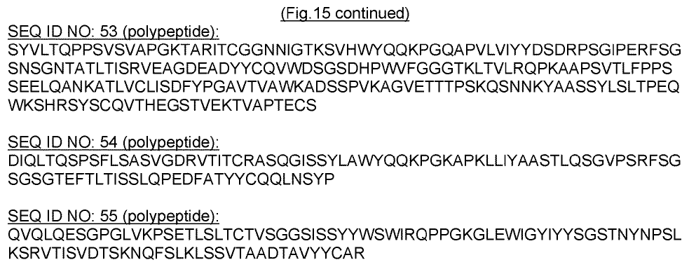

Coadministration of BTK-inhibitors like ibrutinib-a BTK/ITK inhibitor known to restore T cell functionality—to CAR T cell can have additive anti-tumor effects. To test this in the R110 CAR T setting, ibrutinib toxicity for OCI-Ly1 target cells was assessed first. For this, 2×104 OCI-Ly1 wt and OCI-Ly1_R110 cells were seeded in 200 μl culture media supplemented with serial dilutions of ibrutinib (PCI-32765, Selleck Chemicals, Cat. S2680) ranging from 0.01 to 20 μM. Cells grown under the same conditions without ibrutinib served as control. After 24h incubation, viable (trypan blue exclusion) cells were counted using the Vi-CELL XR system (Beckman Coulter). Cell growth in percent was then plotted as total numbers of ibrutinib-treated cells relative to the total number of untreated cells (FIG. 10). This analysis revealed 0.08 UM ibrutinib as highest dose that does not result in a viability drop of target cells. This dose was thus chosen for further testing of CAR T efficacy.

Comparative cytotoxicity assays with humanized anti-IGLV3-21 R110 CAR-T cells derived from healthy donor and IGLV3-21^{R110}-positive CCL patient (CLL425) were performed according to Example 3. For the IncuCyte@ killing assays in the presence of BTK-inhibitor, 0.08 UM Ibrutinib were added per well at seeding of target and effector cells (day 0) for the duration of the assay (16h).

As shown in FIG. 11, the addition of ibrutinib in subtherapeutic dose raised the killing capacity of the CLL derived CAR T cells to the level of CAR-T cells derived from healthy donor.

### Example 5

### Interferon-Gamma (IFN γ) Profile

Immune effector cytokine IFN-gamma was checked during cytotoxicity assays. IFN-gamma concentrations were measured with the bead-based immunoassay technology LEGENDplex™ Human B Cell Panel Standard (BioLegend, Cat. 740537) in 8 to 16 or 36 hours culture supernatant upon co-culture of effector cells with target cells following the manufacturer's protocol. Per well, 25 μL of supernatant was used. Fluorescence was measured on a BD FACSCelesta™ instrument. Values below the limit of detection were considered zero.

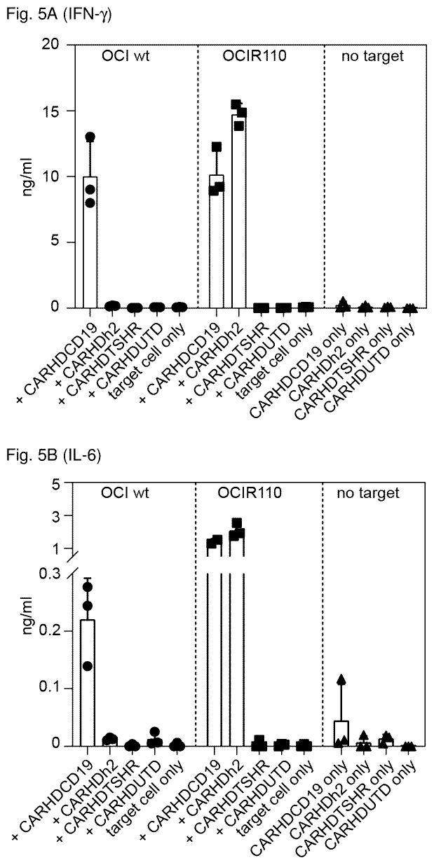

As shown in FIGS. 5A, 8A, and 12, consistent with the cytotoxicity assay results, elevated IFN γ levels were measurable in the respective supernatants that showed cell killing by CAR T cells.

### Example 6

### Interleukin 6 (IL-6) Profile

Immune effector cytokine IL-6 was checked during cytotoxicity assays. IL-6 concentrations were measured with the bead-based immunoassay technology LEGENDplex™ Human B Cell Panel Standard (BioLegend, Cat. 740537) in 8 to 36 hours culture supernatant upon co-culture of effector cells with target cells following the manufacturer's protocol. Per well, 25 μL of supernatant was used. Fluorescence was measured on a BD FACSCelesta™ instrument. Values below the limit of detection were considered zero.

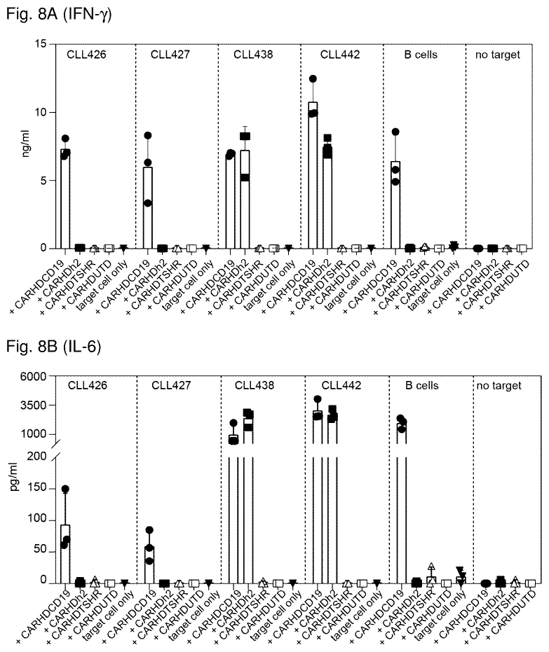

As shown in FIGS. 5B and 8B, consistent with the cytotoxicity assay results, elevated IL-6 levels were measurable in the respective supernatants that showed cell killing by CAR T cells.

1. A humanized chimeric antigen receptor (CARs) comprising: a humanized IGLV3-21^{R110} binding domain, a transmembrane domain, and a cytoplasmic domain,
wherein the humanized IGLV3-21^{R110} binding domain comprises a light chain variable region (VL) which comprises a light chain complementarity-determining region 1 (L-CDR1) having an amino acid sequence of SEQ ID NO: 1, a light chain complementarity-determining region 2 (L-CDR2) having an amino acid sequence of SEQ ID NO: 2, and a light chain complementarity-determining region 3 (L-CDR3) having an amino acid sequence of SEQ ID NO: 3 and a heavy chain variable region (VH) which comprises a heavy chain complementarity-determining region 1 (H-CDR1) having an amino acid sequence of SEQ ID NO: 4, a heavy chain complementarity-determining region 2 (H-CDR2) having an amino acid sequence of SEQ ID NO: 5, and a heavy chain complementarity-determining region 3 (H-CDR3) having an amino acid sequence of SEQ ID NO: 6.

2. The humanized chimeric antigen receptor according to claim 1, wherein the VL has an amino acid sequence selected from SEQ ID NO: 7, SEQ ID NO: 8, SEQ ID NO: 9, and SEQ ID NO: 10 in any combination and the VH has an amino acid sequence selected from SEQ ID NO: 11 and SEQ ID NO: 12.

3. The humanized chimeric antigen receptor according to claim 2, wherein
the VL has an amino acid sequence of SEQ ID NO: 7 and the VH has an amino acid sequence of SEQ ID NO: 11, or
the VL has an amino acid sequence of SEQ ID NO: 7 and the VH has an amino acid sequence of SEQ ID NO: 12.

4. The humanized chimeric antigen receptor according to claim 1, wherein the VL has an amino acid sequence identity of at least 70%, 75%, 80%, 85%, 90%, 95%, 98% to a VL having an amino acid sequence of SEQ ID NO: 7 and an amino acid sequence identity of at least 70%, 75%, 80%, 85%, 90%, 95%, 98% to a VH having an amino acid sequence of SEQ ID NO: 11 or of SEQ ID NO: 12.

5. The humanized chimeric antigen receptor according to claim 1, wherein the humanized anti-IGLV3-21^{R110} binding domain is a scFv.

6. The humanized chimeric antigen receptor according to claim 5, wherein the scFv has an amino acid sequence of SEQ ID NO: 13, SEQ ID NO: 14, SEQ ID NO: 15, SEQ ID NO: 16, SEQ ID NO: 17, SEQ ID NO: 18, SEQ ID NO: 19, or SEQ ID NO: 20.

7. A polynucleotide which encodes a CAR according to claim 1.

8. A vector which comprises a polynucleotide according to claim 7.

9. A cell expressing a CAR according to claim 1.

10. A cell according to claim 9, wherein the cell is a T cell.

11. A pharmaceutical composition comprising a CAR according to claim 1 and a pharmaceutically acceptable carrier.

12. A method of treating a disease comprising administering to a subject a humanized chimeric antigen receptor according to claim 1.

13. A method for treating a disease, comprising administering a CAR according to claim 1 to a subject in need thereof.

14. A method for treating a disease comprising:
i) providing a population of immune cells,
ii) introducing into the immune cells a polynucleotide according to claim 7,
iii) culturing the immune cells under conditions allowing for expression of the CAR, and
iv) administering a cell from (iii) to a subject.

15. The method according to claim 13, wherein the disease is chronic lymphocytic leukemia (CLL).

16. The method according to claim 13, wherein the CAR is administered in combination with an additional therapeutic agent.

17. The method according to claim 16, wherein the additional therapeutic agent is a bruton's tyrosine kinase (BTK) inhibitor.

18. A kit comprising a pharmaceutical composition according to claim 11.

19. A pharmaceutical composition comprising a polynucleotide according to claim 7 and a pharmaceutically acceptable carrier.

20. A pharmaceutical composition comprising a vector according to claim 8 and a pharmaceutically acceptable carrier.
